(19) United States
(12) Patent Application Publication
(10) Pub. No.: US 2024/0009372 A1
(43) Pub. Date: Jan. 11, 2024

(54) CANISTER FOR A NEGATIVE PRESSURE WOUND THERAPY SYSTEM

(71) Applicant: Smith & Nephew, Inc., Memphis, TN (US)

(72) Inventors: Richard M. Braga, North Easton, MA (US); David E. Fink, Franklin, MA (US); David Heagle, Taunton, MA (US); Arnaz S. Malhi, Watertown, MA (US); David R. Swisher, St. Charles, MO (US); Scott Wudyka, Marlborough, MA (US)

(21) Appl. No.: 18/217,432

(22) Filed: Jun. 30, 2023

Related U.S. Application Data
(63) Continuation of application No. 16/102,584, filed on Aug. 13, 2018, now abandoned.
Application No. 16/102,584 is a continuation-in-part of application No. 15/965,732, filed on Apr. 27, 2018, now abandoned.
Application No. 15/965,732 is a continuation of application No. 14/043,529, filed on Oct. 1, 2013, now Pat. No. 9,956,325.
Application No. 14/043,529 is a continuation of application No. 13/476,255, filed on May 21, 2012, now Pat. No. 8,568,386.
Application No. 13/476,255 is a continuation of application No. 12/463,563, filed on May 11, 2009, now Pat. No. 8,251,979.
Application No. 16/102,584 is a continuation-in-part of application No. 15/791,245, filed on Oct. 23, 2017, now Pat. No. 10,737,000.
Application No. 15/791,245 is a continuation of application No. 15/219,045, filed on Jul. 25, 2016, now Pat. No. 9,801,984.
Application No. 15/219,045 is a continuation of application No. 14/473,063, filed on Aug. 29, 2014, now Pat. No. 9,415,145.
Application No. 14/473,063 is a continuation of application No. 12/471,789, filed on May 26, 2009, now Pat. No. 8,827,983.
Application No. 16/102,584 is a continuation-in-part of application No. 14/197,055, filed on Mar. 4, 2014, now abandoned.
Application No. 14/197,055 is a continuation of application No. 13/488,575, filed on Jun. 5, 2012, now Pat. No. 8,679,081.
Application No. 13/488,575 is a continuation of application No. 12/472,465, filed on May 27, 2009, now Pat. No. 8,216,198.
(60) Provisional application No. 61/090,782, filed on Aug. 21, 2008.
(60) Provisional application No. 61/143,449, filed on Jan. 9, 2009.

(51) Int. Cl. A61M 1/00

(52) U.S. Cl. CPC A61M 1/78; A61M 1/73; A61M 1/74; A61M 1/784; A61M 1/882; A61M 1/96; A61M 1/98; A61M 1/982; A61M 1/60; A61M 2205/8206; A61M 2205/7518; A61M 2205/3382; A61M 2205/21; A61M 2205/3317; A61M 2205/7536; A61M 1/75; A61M 1/984

(57) ABSTRACT

A portable negative pressure wound therapy system includes a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister has a filter assembly having a filter and a passageway between the filter and a wall of the collection canister. The collection canister also includes a canister interface having a suction port, an inlet port, and a channel. The vacuum source draws air through the suction port from the channel which draws air from the passageway connected to the channel, the air in the passageway is drawn from the collection canister through the filter, and the air in the collection canister is drawn through the inlet port.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application is also a continuation-in-part of co-pending U.S. application Ser. No. 15/965,732, filed Apr. 27, 2018, which is a continuation of U.S. application Ser. No. 14/043,529, filed Oct. 1, 2013 and issued as U.S. Pat. No. 9,956,325, which is a continuation of U.S. application Ser. No. 13/476,255, filed May 21, 2012 and issued as U.S. Pat. No. 8,568,386, which is a continuation of U.S. application Ser. No. 12/463,563, filed May 11, 2009 and issued as U.S. Pat. No. 8,251,979.

This application is also a continuation-in-part of co-pending U.S. application Ser. No. 15/791,245, filed Oct. 23, 2017, which is a continuation of U.S. application Ser. No. 15/219,045, filed Jul. 25, 2016 and issued as U.S. Pat. No. 9,801,984, which is a continuation of U.S. application Ser. No. 14/473,063, filed Aug. 29, 2014 and issued as U.S. Pat. No. 9,415,145, which is a continuation of U.S. application Ser. No. 12/471,789, filed May 26, 2009 and issued as U.S. Pat. No. 8,827,983, which claims priority to U.S. Provisional Patent Application Ser. No. 61/090,782 filed on Aug. 21, 2008.

This application is also a continuation-in-part of co-pending U.S. application Ser. No. 14/197,055, filed Mar. 4, 2014, which is a continuation of U.S. application Ser. No. 13/488,575, filed on Jun. 5, 2012 and issued as U.S. Pat. No. 8,679,081, which is a continuation of U.S. application Ser. No. 12/472,465, filed on May 27, 2009 and issued as U.S. Pat. No. 8,216,198, which claims priority to U.S. Provisional Patent Application Ser. No. 61/143,449 filed on Jan. 9, 2009.

The disclosures of all these prior applications are hereby incorporated herein by reference in their entireties and are to be to be considered a part of this specification.

### BACKGROUND OF THE INVENTION

### Field of the Invention

In some embodiments, the present disclosure relates generally to treating a wound by applying negative pressure to the wound, and, more specifically, to a canister for use with a negative pressure wound therapy system that is operable in any orientation.

In some embodiments, the present disclosure relates to negative pressure wound therapy systems and, more particularly, to a sensor with electrical contact protection for use in a fluid collection canister and negative pressure wound therapy systems including the same.

In some embodiments, the present disclosure relates to treating an open wound with a wound therapy system, and, more specifically, relates to a collection canister for a wound therapy system incorporating a baffle mechanism for minimizing flow of exudates within the canister thereby reducing the potential of spillage and communication of the exudates with operating components of the wound therapy system.

### Description of the Related Art

Wound closure involves the migration of epithelial and subcutaneous tissue adjacent the wound towards the center and away from the base of the wound until the wound closes. Unfortunately, closure is difficult with large wounds, chronic wounds or wounds that have become infected. In such wounds, a zone of stasis (i.e. an area in which localized swelling of tissue restricts the flow of blood to the tissues) forms near the surface of the wound. Without sufficient blood flow, the epithelial and subcutaneous tissues surrounding the wound not only receive diminished oxygen and nutrients, but, are also less able to successfully fight microbial infection and, thus, are less able to close the wound naturally. Such wounds have presented difficulties to medical personnel for many years.

Negative pressure wound therapy (NPWT), also known as suction or vacuum therapy, has been used in treating and healing wounds. Treating an open wound by application of negative pressure, e.g. reduced or sub-atmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound such as exudates, which may tend to harbor bacteria. Negative pressure therapy can thus be applied as a healing modality for its antiseptic and tissue regeneration effects. This technique has proven particularly effective for treating a variety of wound conditions, including chronic or healing-resistant wounds and ulcers, and is also used for other purposes such as post-operative wound care.

Generally, negative pressure therapy provides for a wound covering to be positioned over the wound to facilitate suction at the wound area. A conduit is introduced through the wound covering to provide fluid communication to an external vacuum source, such as a hospital vacuum system or a portable vacuum pump. Atmospheric gas, wound exudates, or other fluids may thus be drawn from the reservoir through the fluid conduit to stimulate healing of the wound. Generally, a fluid collection canister for collecting fluids aspirated from the wound is positioned in the suction line between the wound covering and the vacuum source. Exudates drawn from the reservoir through the fluid conduit may thus may be deposited in a collection canister, which may be disposable.

Often, a portable NPWT device is worn by the patient so that the patient may remain ambulatory instead of being confined to a stationary position. While a patient is ambulatory, the portable NPWT device tends to tip or tilt in a multitude of directions. If there are enough exudates in the collection canister, the exudates may cover a suction port leading from the vacuum source to the collection canister because fluid seeks its own level. Covering the suction port prevents the application of negative pressure to the wound thereby discontinuing wound therapy. Additionally, covering the suction port may provide a false indication that the collection canister is full and needs to be replaced when there may be additional space in the canister to fill with exudate.

In addition, portable NPWT devices have a control unit attached to the canister. The control unit generally contains the suction pump and sensitive electronics such as a pressure transducers, microprocessors, or the like. When the NPWT device tips, exudate may aspirate from the canister into the control unit thereby damaging the suction pump and/or electronic components.

The fluid collection canister of the wound therapy system may need to be disconnected or replaced for a variety of reasons, such as when filled with exudate. A mechanism for preventing overfilling of the collection canister may prevent fluid contamination of various components of the negative pressure wound therapy system and help to prevent spillage or leakage. During a treatment, the collection canister may be prevented from overfilling by a hydrophobic filter at the top of the collection canister that shuts off the air flow to the vacuum source when the collection canister is full. During some treatments, the collection canister is replaced or emptied of exudate on a regular scheduled basis, e.g., every few days or so. The collection canister may fill more quickly than anticipated. If this occurs, therapy cannot be delivered to the wound until the collection canister is emptied or replaced. There is a need for a negative pressure wound therapy system that is capable of providing an indication to alert the user that the collection canister must be emptied or replaced.

The collection canister fills with exudates and is typically changed every few days. The collected exudates are free to move around within the canister and may cause vacuum delay, such as by accidental contact with fluid sensors or other electronics of the system. Movement of the canister exudates may also cause spillage from the collection canister which may cause exposure to the collected wound exudates or affect disposal of the collection canister. Furthermore, varying species of bacteria and fungal isolates within the exudates grow and proliferate, which ultimately leads to foul odor. It would be advantageous to provide a collection canister with a baffle mechanism contained therein to reduce movement of exudates, to aid in disposal, and to limit any odor produced therein.

### SUMMARY OF THE INVENTION

In some embodiments, the present disclosure relates to a portable NPWT system including a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister has a filter assembly having a first filter and a second filter at opposing ends of the collection canister. There is a first passageway between the first filter and a wall of the collection canister and a second passageway between the second filter and a wall of the collection canister. The collection canister also includes a canister interface having a suction port, an inlet port, and a channel. The vacuum source draws air through the suction port from the channel which draws air from the passageway connected to the channel, the air in the passageway is drawn from the collection canister through the filter, and the air in the collection canister is drawn through the inlet port.

In some embodiments, the present disclosure also relates to a portable NPWT system including a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister includes a filter assembly having at least one filter, a passageway between the filter and a wall of the collection canister, and a canister interface having a suction port, an inlet port, and a channel. The vacuum source draws air through the suction port from the channel which draws air from the passageway connected to the channel. The air in the passageway is drawn from the collection canister through the filter and the air in the collection canister is drawn through the inlet port.

In some embodiments, the present disclosure relates to a sensor for use in a canister for fluid collection, the canister having a canister top and defining a fluid collection chamber. The sensor includes a first electrode and a second electrode. The first electrode includes a first portion and a second portion, wherein the first portion of the first electrode is supported by the canister top, and the second portion of the first electrode is configured to extend into the fluid collection chamber. The second electrode includes a first portion and a second portion, wherein the first portion of the second electrode is supported by the canister top, and the second portion of the second electrode is configured to extend into the fluid collection chamber. The sensor also includes an electric circuit configured to detect an electrical property associated with the first and second electrodes. The second portions of the first and second electrodes may be at least partly covered with a coating to inhibit encrustation formed by drying of exudate. The sensor may also include an inner chamber disposed within the fluid collection chamber, the inner chamber bounded by the canister top, an inner wall and a bottom end, wherein the second portions of the first and second electrodes are disposed within the inner chamber. At least a portion of the bottom end of the inner chamber may be formed of a water-soluble film.

In some embodiments, the present disclosure also relates to a portable negative pressure wound therapy apparatus including a dressing assembly for positioning over a wound to apply a negative pressure to the wound and a canister assembly in fluid communication with the dressing assembly. The canister assembly includes a control unit, a vacuum source disposed in the control unit, a pressure sensor in communication with the control unit, and a collection canister. The collection canister includes an inlet conduit in fluid communication with the dressing assembly, a chamber to collect wound fluids from the dressing assembly, an inlet port coupled to the inlet conduit to introduce the wound fluids from the dressing assembly into the chamber, and a suction port to communicate with the chamber and the vacuum source. The canister assembly may also include a transducer port to communicate with the chamber and the pressure sensor.

In some embodiments, the present disclosure also relates to a system for negative pressure therapy in connection with healing a wound including a dressing assembly for positioning relative to a wound bed and a negative pressure mechanism. The negative pressure mechanism includes a control unit, a collection canister for collecting exudate removed from the wound bed under negative pressure supplied by the control unit, and a detector circuit. The detector circuit includes first and second electrically conductive contacts disposed within the collection canister and an indicator adapted to provide a perceptible indication when exudate makes contact with the first and second electrically conductive contacts. The detector circuit is open in the absence of exudate making contact with the first and second electrically conductive contacts. The detector circuit is closed when exudate makes contact with the first and second electrically conductive contacts. The first and second electrically conductive contacts may be mounted at a position within the collection canister corresponding to a maximum fill volume of the collection canister.

In some embodiments, the present disclosure relates to a system to promote the healing of an exuding wound includes a wound dressing, a subatmospheric pressure mechanism, and a collection canister. The wound dressing is dimensioned for positioning relative to a wound bed of a subject. The subatmospheric pressure mechanism includes a control unit disposed within a housing. The control unit includes a vacuum source associated with a vacuum port. The collection canister has an interior wall which defines an internal chamber. The internal chamber is in fluid communication with the vacuum source of the subatmospheric pressure mechanism through the vacuum port and with the wound dressing for collecting exudates removed for the wound bed under influence of the vacuum source. The canister including a baffle mechanism disposed within the internal chamber for dampening the movement of the collected exudates.

The baffle mechanism increases the surface area within the canister. The additional surfaces serve to resist motion of the exudates upon movement of the canister and absorb the kinetic energy of the moving exudates. In embodiments, the baffle mechanism includes at least one baffle in the form of a permeable material segment. The material segment is treated with an antimicrobial agent to allow passage and treatment of the exudates therethrough for controlling the growth of microorganisms in the exudates. In embodiments, a plurality of baffles depends from the interior wall and within the internal chamber of the collection canister. Groups of baffles are similarly oriented within the canister to assist in impeding flow of the exudates. In yet other embodiments, a fluid solidifier is disposed in a select portion of the canister and maintained in place via a water soluble adhesive for dampening movement of exudates by consolidating the exudates into a substantially non-flowable state.

### BRIEF DESCRIPTION OF THE DRAWINGS

Objects and features of the presently disclosed negative pressure wound therapy systems and sensors for use therein will become apparent to those of ordinary skill in the art when descriptions of various embodiments thereof are read with reference to the accompanying drawings. Various embodiments of the wound dressing system of the present disclosure are described herein with reference to the drawings wherein:

FIG. 1 is a diagram of a NPWT system in accordance with an embodiment of the present disclosure;

FIG. 2 is a diagram of a NPWT system in accordance with an embodiment of the present disclosure;

FIG. 3 is a diagram of a canister for a NPWT system in accordance with an embodiment of the present disclosure;

FIG. 4 is a diagram of a canister for a NPWT system in accordance with an embodiment of the present disclosure;

FIG. 5A is a diagram of a canister for a NPWT system in accordance with an embodiment of the present disclosure;

FIG. 5B is a diagram of a canister for a NPWT system in accordance with an embodiment of the present disclosure;

FIG. 6 is a diagram of a canister assembly for an NPWT system in accordance with an embodiment of the present disclosure;

FIG. 7A is a top view of a canister for a NPWT system in accordance with an embodiment of the present disclosure;

FIG. 7B is a top view of a canister interface for a NPWT system in accordance with an embodiment of the present disclosure;

FIG. 8A is a top view of a canister for a NPWT system in accordance with an embodiment of the present disclosure;

FIG. 8B is a top view of a canister interface for a NPWT system in accordance with an embodiment of the present disclosure;

FIG. 9A is a top view of a canister for a NPWT system in accordance with an embodiment of the present disclosure;

FIG. 9B is a top view of a canister interface for a NPWT system in accordance with an embodiment of the present disclosure;

FIG. 10 is a schematic diagram of the canister assembly of the negative pressure wound therapy system illustrated in FIG. 2;

FIG. 11 is a cross-sectional view of the collection canister of the canister assembly shown in FIG. 2 taken along the lines 4-4;

FIG. 12 is a cross-sectional view of the collection canister shown in FIG. 4 taken along the lines 5-5;

FIG. 13 is a bottom view of the collection canister top shown in FIG. 4;

FIG. 14 is a cross-sectional view of another embodiment of a collection canister in accordance with the present disclosure;

FIG. 15 is a cross-sectional view of yet another embodiment of a collection canister in accordance with the present disclosure;

FIGS. 16A through 16C are perspective views of embodiments of the electrical contact protection unit of the collection canister illustrated in FIG. 8, shown with varied geometric shapes, in accordance with the present disclosure;

FIG. 17 is a schematic diagram of another embodiment of a collection canister in accordance with the present disclosure;

FIG. 18 is a cross-sectional view of yet another embodiment of a collection canister in accordance with the present disclosure;

FIG. 19 is a perspective view of the canister assembly of the negative pressure wound therapy system illustrated in FIG. 2 shown with a window having fluid level markings in accordance with the present disclosure;

FIG. 20 is a schematic diagram of an embodiment of a detector circuit in accordance with the present disclosure;

FIG. 21A is a schematic diagram of an embodiment of a detection circuit in accordance with the present disclosure;

FIG. 21B is a schematic diagram of another embodiment of a detection circuit in accordance with the present disclosure;

FIG. 22 is a view in partial cross-section of a wound therapy system of the present disclosure illustrating the wound dressing, the subatmospheric pressure mechanism, and the collection canister;

FIG. 23 is a perspective cross-sectional view of an embodiment of a subatmospheric pressure mechanism and a canister of the wound therapy system of the present disclosure;

FIG. 24 is a cross-sectional view of a collection canister having a baffle mechanism including an antimicrobial treated material segment contained therein in accordance with the present disclosure;

FIG. 25 is a cross-sectional view of another embodiment of a collection canister including an antimicrobial treated material segment contained therein;

FIGS. 26A-26B are side and top views of another embodiment of an antimicrobial treated material segment of a baffle mechanism of the collection canister of the present disclosure;

FIG. 27 is a top view of an embodiment of a collection canister having a baffle mechanism including a plurality of baffles contained therein in accordance with the present disclosure;

FIG. 28 is a side perspective view in partial cross-section of the collection canister of FIG. 27;

FIG. 29 is a side cross-sectional view of an embodiment of a collection canister having a baffle mechanism including a fluid solidifier coated therein in accordance with the present disclosure; and

FIG. 30 is a side cross-sectional view of another embodiment of a collection canister including a fluid solidifier contained therein.

### DETAILED DESCRIPTION OF THE PREFERRED EMBODIMENT

The wound therapy system of the present disclosure promotes healing of a wound via the use of a wound dressing, a subatmospheric pressure mechanism, and a collection canister. Generally, the subatmospheric pressure mechanism applies subatmospheric pressure to the wound to effectively remove wound fluids or exudates captured within the boundary of the composite wound dressing, and to increase blood flow to the wound bed and enhance cellular stimulation of epithelial and subcutaneous tissue. The wound therapy system may be entirely portable, i.e., it may be worn or carried by the subject such that the subject may be completely ambulatory during the therapy period. The wound therapy system including the subatmospheric pressure mechanism and components thereof may be entirely reusable or may be entirely disposable after a predetermined period of use or may be individually disposable whereby some of the components are reused for a subsequent therapy application.

The wound therapy system of the present disclosure promotes healing of a wound in conjunction with subatmospheric negative pressure therapy. The system may incorporate a variety of wound dressings, subatmospheric pressure sources and pumps, and collection canisters. The attached figures illustrate exemplary embodiments of the present disclosure and are referenced to describe the embodiments depicted therein. Hereinafter, the disclosure will be described by explaining the figures wherein like reference numerals represent like parts throughout the several views.

Various embodiments of the present disclosure provide negative pressure wound therapy systems (or apparatus) including a collection canister having a chamber to collect wound fluids. Embodiments of the presently disclosed negative pressure wound therapy systems are generally suitable for use in applying negative pressure to a wound to facilitate healing of the wound in accordance with various treatment modalities. Embodiments of the presently disclosed negative pressure wound therapy systems are entirely portable and may be worn or carried by the user such that the user may be completely ambulatory during the therapy period. Embodiments of the presently disclosed negative pressure wound therapy apparatus and components thereof may be entirely reusable or may be entirely disposable after a predetermined period of use or may be individually disposable whereby some of the components are reused for a subsequent therapy application.

Hereinafter, embodiments of the presently disclosed negative pressure wound therapy systems and embodiments of the presently disclosed sensors for use in negative pressure wound therapy systems will be described with reference to the accompanying drawings. Like reference numerals may refer to similar or identical elements throughout the description of the figures. As used herein, “wound exudate”, or, simply, “exudate”, generally refers to any fluid output from a wound, e.g., blood, serum, and/or pus, etc. As used herein, “fluid” generally refers to a liquid, a gas or both. As used herein, “transmission line” generally refers to any transmission medium that can be used for the propagation of signals from one point to another.

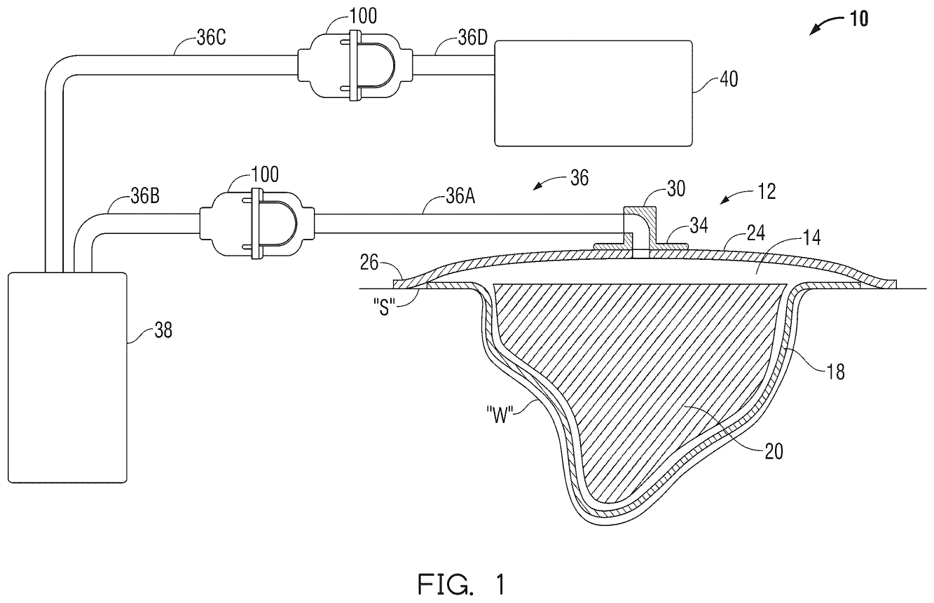

Referring to FIG. 1, a negative pressure wound therapy apparatus according to an embodiment of the present disclosure is depicted generally as 10 for use on a wound bed “w” surrounded by healthy skin “s”. Negative pressure wound therapy apparatus 10 includes a wound dressing 12 positioned relative to the wound bed “w” to define a vacuum chamber 14 about the wound bed “w” to maintain negative pressure at the wound area. Wound dressing 12 includes a contact layer 18, a wound filler 20 and a wound cover 24.

Contact layer 18 is intended for placement within the wound bed “w” and may be relatively non-supportive or flexible to substantially conform to the topography of the wound bed “w”. A variety of materials may be used for the contact layer 18. Contact layer 18 selection may depend on various factors such as the patient's condition, the condition of the periwound skin, the amount of exudate and/or the condition of the wound bed “w”. Contact layer 18 may be formed from perforated film material. The porous characteristic of the contact layer 18 permits exudate to pass from the wound bed “w” through the contact layer 18. Passage of wound exudate through the contact layer 18 may be substantially unidirectional such that exudate does not tend to flow back into the wound bed “w”. Unidirectional flow may be encouraged by directional apertures, e.g., apertures positioned at peaks of undulations or cone-shaped formations protruding from the contact layer 18. Unidirectional flow may also be encouraged by laminating the contact layer 18 with materials having absorption properties differing from those of the contact layer 18, or by selection of materials that promote directional flow. A non-adherent material may be selected for forming the contact layer 18 such that the contact layer 18 does not tend to cling to the wound bed “w” or surrounding tissue when it is removed. One example of a material that may be suitable for use as a contact layer 18 is commercially available under the trademark XEROFLOW® offered by Tyco Healthcare Group LP (d/b/a Covidien). Another example of a material that may be suitable for use as the contact layer 18 is the commercially available CURITY® non-adherent dressing offered by Tyco Healthcare Group LP (d/b/a Covidien).

Wound filler 20 is positioned in the wound bed “w” over the contact layer 18 and is intended to transfer wound exudate. Wound filler 20 is conformable to assume the shape of any wound bed “w” and may be packed up to any level, e.g., up to the level of healthy skin “s” or to overfill the wound such that wound filler 20 protrudes over healthy skin “s”. Wound filler 20 may be treated with agents such as polyhexamethylene biguanide (PHMB) to decrease the incidence of infection and/or other medicaments to promote wound healing. A variety of materials may be used for the wound filler 20. An example of a material that may be suitable for use as the wound filler 20 is the antimicrobial dressing commercially available under the trademark KERLIX™ AMD offered by Tyco Healthcare Group LP (d/b/a Covidien).

Cover layer 24 may be formed of a flexible membrane, e.g., a polymeric or elastomeric film, which may include a biocompatible adhesive on at least a portion of the cover layer 24, e.g., at the periphery 26 of the cover layer 24. Alternately, the cover layer 24 may be a substantially rigid member. Cover layer 24 may be positioned over the wound bed “w” such that a substantially continuous band of a biocompatible adhesive at the periphery 26 of the cover layer 24 forms a substantially fluid-tight seal with the surrounding skin “s”. An example of a material that may be suitable for use as the cover layer 24 is commercially available under the trademark CURAFORM ISLAND® offered by Tyco Healthcare Group LP (d/b/a Covidien).

Cover layer 24 may act as both a microbial barrier and a fluid barrier to prevent contaminants from entering the wound bed “w” and to help maintain the integrity thereof.

In one embodiment, the cover layer 24 is formed from a moisture vapor permeable membrane, e.g., to promote the exchange of oxygen and moisture between the wound bed “w” and the atmosphere. An example of a membrane that may provide a suitable moisture vapor transmission rate (MVTR) is a transparent membrane commercially available under the trade name POLYSKIN® II offered by Tyco Healthcare Group LP (d/b/a Covidien). A transparent membrane may help to permit a visual assessment of wound conditions to be made without requiring removal of the cover layer 24.

Wound dressing 12 may include a vacuum port 30 having a flange 34 to facilitate connection of the vacuum chamber 14 to a vacuum system. Vacuum port 30 may be configured as a rigid or flexible, low-profile component and may be adapted to receive a conduit 36 in a releasable and fluid-tight manner. An adhesive on at least a portion of the underside of the flange 34 may be used to provide a mechanism for affixing the vacuum port 30 to the cover layer 24. The relative positions, size and/or shape of the vacuum port 30 and the flange 34 may be varied from an embodiment depicted in FIG. 1. For example, the flange 34 may be positioned within the vacuum chamber 14 such that an adhesive on at least a portion of an upper side surface of the flange 34 affixes the vacuum port 30 to the cover layer 24. A hollow interior portion of the vacuum port 30 provides fluid communication between the conduit 36 and the vacuum chamber 14. Conduit 36 extends from the vacuum port 30 to provide fluid communication between the vacuum chamber 14 and the vacuum source 40. Alternately, the vacuum port 30 may not be included in the dressing 12 if other provisions are made for providing fluid communication with the conduit 36.

Any suitable conduit may be used for the conduit 36, including conduit fabricated from flexible elastomeric or polymeric materials. In the negative pressure wound therapy apparatus 10 illustrated in FIG. 1, the conduit 36 includes a first conduit section 36A, a second conduit section 36B, a third conduit section 36C and a fourth conduit section 36D. The first conduit section 36A extends from the vacuum port 30 and is coupled via a fluid line coupling 100 to the second conduit section 36B, which extends to the collection canister 38. The third conduit section 36C extends from the collection canister 38 and is coupled via another fluid line coupling 100 to the fourth conduit section 36D, which extends to the vacuum source 40. The shape, size and/or number of conduit sections of the conduit 36 may be varied from the first, second, third and fourth conduit sections 36A, 36B, 36C and 36D depicted in FIG. 1.

The first, second, third and fourth conduit sections 36A, 36B, 36C and 36D of the conduit 36 may be connected to components of the apparatus 10 by conventional air-tight means, such as, for example, friction fit, bayonet coupling, or barbed connectors. The connections may be made permanent. Alternately, a quick-disconnect or other releasable connection means may be used to provide some adjustment flexibility to the apparatus 10.

Collection canister 38 may be formed of any type of container that is suitable for containing wound fluids. For example, a semi-rigid plastic bottle may be used for the collection canister 38. A flexible polymeric pouch or other hollow container body may be used for the collection canister 38. Collection canister 38 may contain an absorbent material to consolidate or contain the wound fluids or debris. For example, super absorbent polymers (SAP), silica gel, sodium polyacrylate, potassium polyacrylamide or related compounds may be provided within collection canister 38. At least a portion of canister 38 may be transparent or semi-transparent, e.g., to permit a visual assessment of the wound exudate to assist in evaluating the color, quality and/or quantity of exudate. A transparent or semi-transparent portion of the collection canister 38 may permit a visual assessment to assist in determining the remaining capacity or open volume of the canister and/or may assist in determining whether to replace the collection canister 38.

The collection canister 38 is in fluid communication with the wound dressing 12 via the first and second conduit sections 36A, 36B. The third and fourth conduit sections 36C, 36D connect the collection canister 38 to the vacuum source 40 that generates or otherwise provides a negative pressure to the collection canister 38. Vacuum source 40 may include a peristaltic pump, a diaphragmatic pump or other suitable mechanism. Vacuum source 40 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg to about 125 mmHg, or about 40 mmHg to about 80 mmHg. One example of a peristaltic pump that may be used as the vacuum source 40 is the commercially available KANGAROO PET™ Eternal Feeding Pump offered by Tyco Healthcare Group LP (d/b/a Covidien). Vacuum source 40 may be actuated by an actuator (not shown) which may be any means known by those skilled in the art, including, for example, alternating current (AC) motors, direct current (DC) motors, voice coil actuators, solenoids, and the like. The actuator may be incorporated within the vacuum source 40.

In embodiments, the negative pressure wound therapy apparatus 10 includes one or more fluid line couplings 100 that allow for selectable coupling and decoupling of conduit sections. For example, a fluid line coupling 100 may be used to maintain fluid communication between the first and second conduit sections 36A, 36B when engaged, and may interrupt fluid flow between the first and second conduit sections 36A, 36B when disengaged. Thus, fluid line coupling 100 may facilitate the connection, disconnection or maintenance of components of the negative pressure wound therapy apparatus 10, including the replacement of the collection canister 38. Additional or alternate placement of one or more fluid line couplings 100 at any location in line with the conduit 36 may facilitate other procedures. For example, the placement of a fluid line coupling 100 between the third and fourth conduit sections 36C, 36D, as depicted in FIG. 1, may facilitate servicing of the vacuum source 40.

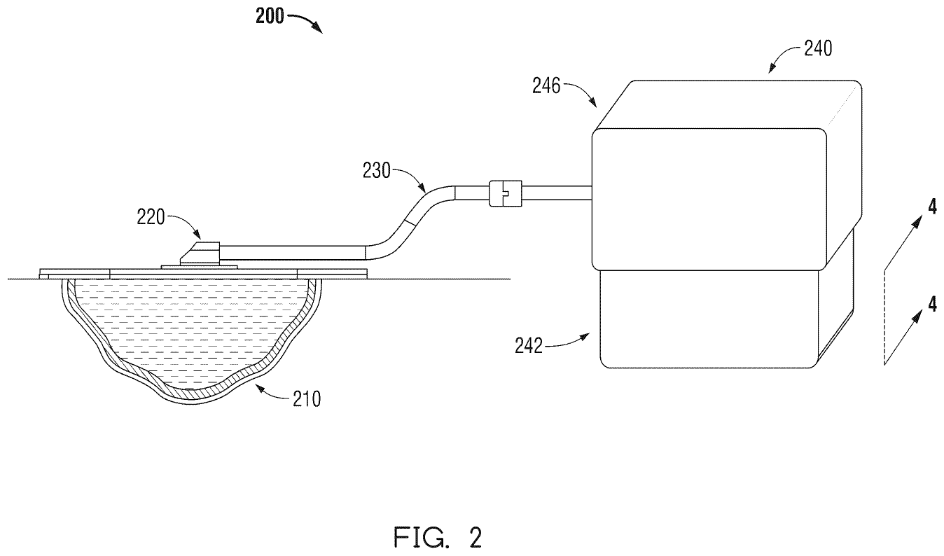

Referring to FIG. 2, the negative pressure wound therapy system shown generally as 200 includes a dressing assembly 210, a wound port assembly 220, an extension assembly 230 and a canister assembly 240. Dressing assembly 210 is positioned relative to the wound area to define a vacuum chamber about the wound area to maintain negative pressure at the wound area. Dressing assembly 210 may be substantially sealed from extraneous air leakage, e.g., using adhesive coverings. Wound port assembly 220 is mounted to the dressing assembly 210. For example, wound port assembly 220 may include a substantially continuous band of adhesive at its periphery for affixing the wound port assembly 220 to the dressing assembly 210. Extension assembly 230 is coupled between the wound port assembly 220 and the canister assembly 240 and defines a fluid flow path between the wound port assembly 220 and the canister assembly 240. A hollow interior of the wound port assembly 220 provides fluid communication between the extension assembly 230 and the interior of the dressing assembly 210. Dressing assembly 210 and the wound port assembly 220 shown in FIG. 2 are similar to components of the wound dressing 12 of FIG. 1 and further description thereof is omitted in the interests of brevity.

Canister assembly 240 includes a control unit 246 and a collection canister 242 disposed below the control unit 246. Control unit 246 and the collection canister 242 may be releasably coupled. Mechanisms for selective coupling and decoupling of the control unit 246 and the collection canister 242 include fasteners, latches, clips, straps, bayonet mounts, magnetic couplings, and other devices. Collection canister 242 may consist of any container suitable for containing wound fluids.

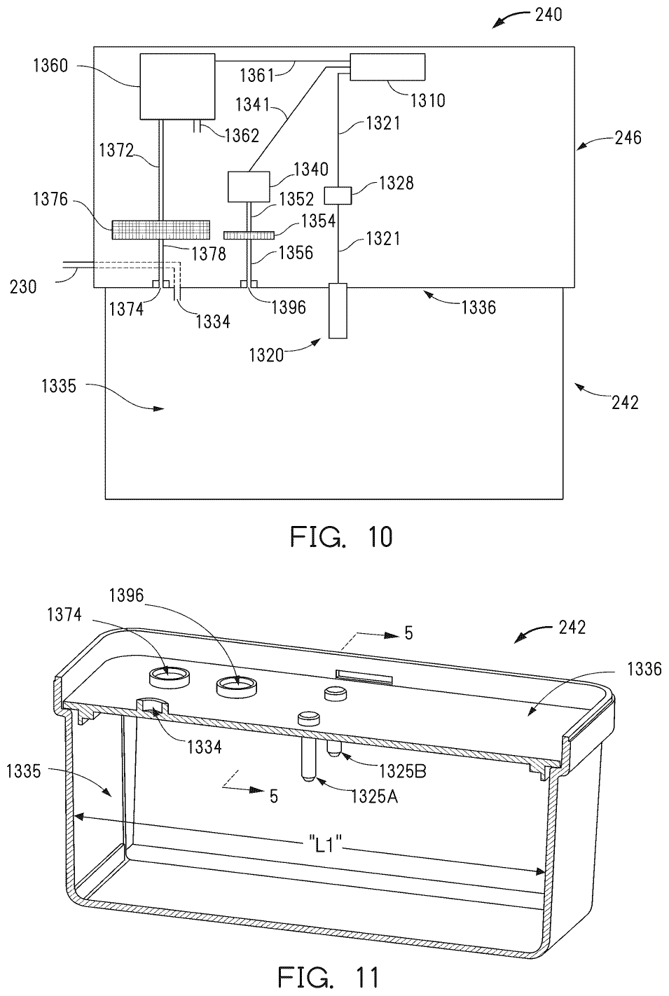

In one embodiment, the negative pressure wound therapy system 200 is capable of operating in a continuous mode or an alternating mode. In the continuous mode, the control unit 246 controls a pump (e.g., suction pump 1360 shown in FIG. 10) to continuously supply a selected vacuum level at the collection canister 242 to create a reduced pressure state within the dressing assembly 210. In the alternating mode, the control unit 246 controls the pump to alternating supply a first negative pressure, e.g., about 80 mmHg, at the collection canister 242 for a preset fixed amount of time and a second negative pressure, e.g., about 50 mmHg, at the collection canister 242 for a different preset fixed amount of time.

In general, the output of the pump is directly related to the degree of air leakage in the negative pressure wound therapy system 200 and the open volume in the collection canister 242. If there is sufficient air leakage in the system 200, e.g., at the dressing assembly 210, the pump can remain on continuously and the control unit 246 can control negative pressure at the collection canister 242 by adjusting the pump speed. Alternatively, if there is not sufficient air leakage in the system 200 to permit the pump to remain on continuously, the control unit 246 can control negative pressure at the collection canister 242 by turning the pump on and off, e.g., for non-equal on/off periods of time.

Control unit 246 responds to various sensed events by signaling alarms. Various types of conditions may be signaled by alarms. In embodiments, control unit 246 is capable of signaling alarms for failed pressure sensor condition, use odometer expired condition, watchdog reset condition, failed pump condition, leak condition, replace canister condition, excessive vacuum condition, failed LEDs condition, low battery condition, very low battery condition, and failed battery condition. Priority levels may be associated with alarms. In embodiments, the priority levels of alarms are low priority alarm, medium priority alarm, and system alarm (highest priority). Low priority alarms, when triggered, may be continuously indicated. Medium priority alarms and system alarms, when triggered, may have a flashing indication.

Control unit 246 may stop operation of the pump (e.g., suction pump 1360 shown in FIG. 10) in response to an alarm, e.g., depending on alarm type and/or priority level. In embodiments, the control unit 246 stops operation of the pump in response to system alarms, e.g., failed pressure sensor system alarm, use odometer expired system alarm, watchdog reset system alarm, failed pump system alarm, excessive vacuum system alarm, and/or failed LEDs system alarm.

If an air leak develops in the negative pressure wound therapy system 200, e.g., at the dressing assembly 210, for which the control unit 246 cannot compensate by increasing the pump speed, the control unit 246 may indicate an alarm. For example, the control unit 246 may indicate a leak alarm after two consecutive minutes of operation in which the vacuum level is below the current set point (or below the minimum level of a set point range).

Audible indicatory means may also be incorporated or associated with the control unit 246 to notify the user of a condition, e.g., leak, canister assembly tip, failed pressure sensor, failed pump, excessive vacuum, or low battery conditions. The audio indication for some alarm types can be paused by pressing a pause alarm button (not shown).

In embodiments, the control unit 246 includes a user interface (not shown). Control unit 246 also includes a processor (e.g., 1310 shown in FIG. 10). A pressure transducer (e.g., transducer 1340 shown in FIG. 10) is electrically coupled to the processor. The user turns ON the canister assembly 240 by pressing a power button (not shown). When the power button is pressed, the control unit 246 performs a series of internal checks during power up. In one embodiment, after successfully completing the power-up tasks, the control unit 246 turns on the pump 1360 using the stored settings. At initial activation of the canister assembly 240, the stored settings are the default settings. In one embodiment, the default settings for controlling the pump 1360 are 80 mmHg and continuous mode. In one embodiment, the currently stored vacuum level setting can be altered by the user, e.g., to 50 mmHg. In one embodiment, the currently stored mode setting can be altered by the user, e.g., to an alternating mode.

Canister assembly 240 may be constructed from a variety of materials such as Lucite™ polycarbonate, metals, metal alloys, plastics, or other durable materials capable of withstanding forces applied during normal use, and may have some capability of withstanding possibly excessive forces resulting from misuse. Collection canister 242 may include a window (e.g., 2223 shown in FIG. 19) with fluid level markings or graduations (e.g., 2225 shown in FIG. 19) for promoting visual assessment of the amount of exudate contained within the collection canister 242. A transparent or partially transparent collection canister 242 may thus assist in determining the remaining capacity of the collection canister 242 and/or when the collection canister 242 should be replaced.

Orientation Independent Canister for a Negative Pressure Wound Therapy Device

A portable negative pressure wound therapy system includes a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister has a filter assembly having a filter and a passageway between the filter and a wall of the collection canister. The collection canister also includes a canister interface having a suction port, an inlet port, and a channel. The vacuum source draws air through the suction port from the channel which draws air from the passageway connected to the channel, the air in the passageway is drawn from the collection canister through the filter, and the air in the collection canister is drawn through the inlet port.

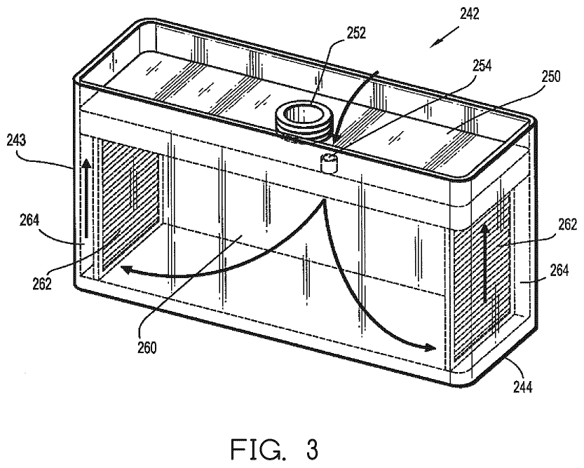

Referring to FIG. 3, a collection canister, shown generally as 242, in accordance with an embodiment of the present disclosure is shown. The collection canister 242 has a canister interface 250 with a suction port 252 and an inlet port 254. Inlet port 254 receives exudate from the wound “w” through the wound port assembly 220 and extension assembly 230. Suction port 252 is coupled to a pump (not shown) in control unit 246. Exudate is collected in chamber or space 260 of the canister 242.

Collection canister 242 also includes a pair of filters 262 on opposing sides of the collection canister 242. Although FIG. 3 shows the filters 262 parallel to the left wall 243 and right wall 244 of the collection canister 242, filters 262 may also be placed parallel to the front and rear walls of the collection canister 242. Filter 262 may be a hydrophobic or oleophobic filter to prevent exudate from exiting the canister. Filter 262 may be a replaceable cartridge that can be replaced during a canister change. Filter 262 may also include an antibacterial coating. Although not shown, an odor filter such as a charcoal filter may also be placed in line with filter 262 to reduce odor produced by exudate. Between filter 262 and the left wall 243 or right wall 244 is a fluid path or passageway 264.

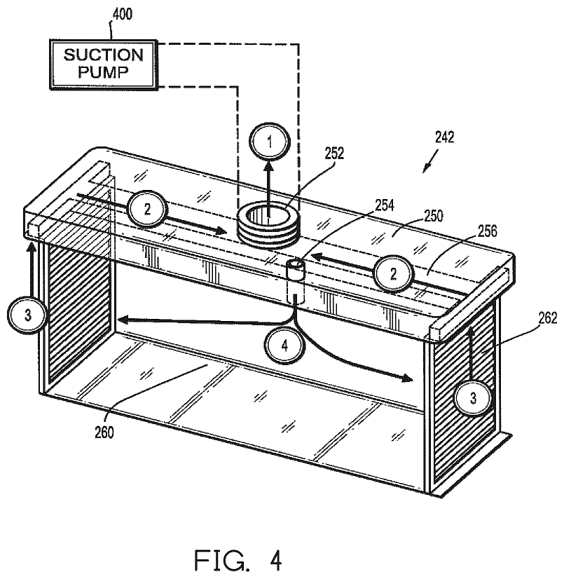

During operation of the NPWT device, the suction pump 400 (as shown in FIG. 4) draws air through suction port 252 in canister interface 250. This causes air to be drawn up passageways 264 from chamber 260 through filters 262. Filters 262 prevent exudate from entering passageway 264 while allowing air to pass through. Drawing air from chamber 260 into passageways 264 imparts a negative pressure in chamber 260 thereby causing air and/or exudate to collect in chamber 260 via inlet port 254.

Referring to FIG. 4, canister 242 is depicted without the external walls of canister 242 and canister interface 250 to show the movement of air within canister 242. FIG. 4 depicts four stages of air movement through the canister. As previously described, suction pump 400 draws in air from suction port 252 as shown in stage 1. In stage 2, air is drawn through channel 256 to the suction port 252. Channel 256 extends from the left side to the right side of the canister interface 250. Below channel 256 along the left edge and the right edge of the canister interface are passageways 264 of FIG. 3. In stage 3, air is drawn from passageways 264 to channel 256. Air in passageways 264 is drawn from chamber 260 as shown in stage 4.

The canister 242 shown in FIGS. 3 and 4 depict an embodiment according to the present disclosure that enables a NPWT device to continue functioning if the canister assembly has been tipped, tilted or inverted. If the canister assembly is tipped or tilted toward the left wall 243 thereby covering the filter 262 on the left wall 243, air is continuously drawn through filter 262 on the right wall 244. Because air is still drawn through the filter 262 along the right wall 244, the NPWT device can maintain a negative or reduced pressure in the chamber 260 thereby maintaining a negative or reduced pressure at the wound “w”. The same holds true if the canister is tipped toward the right wall 244 because the filter 262 along the left wall 243 will remain uncovered. If the canister assembly is inverted, air will still be drawn through filters 262 where exudate has not covered the filters 262. Also, because the suction port 252 is isolated from the chamber 260 where exudate is present by filter 262, passageway 264 and channel 256, exudate cannot enter the control unit 246 thereby damaging the electronics therein.

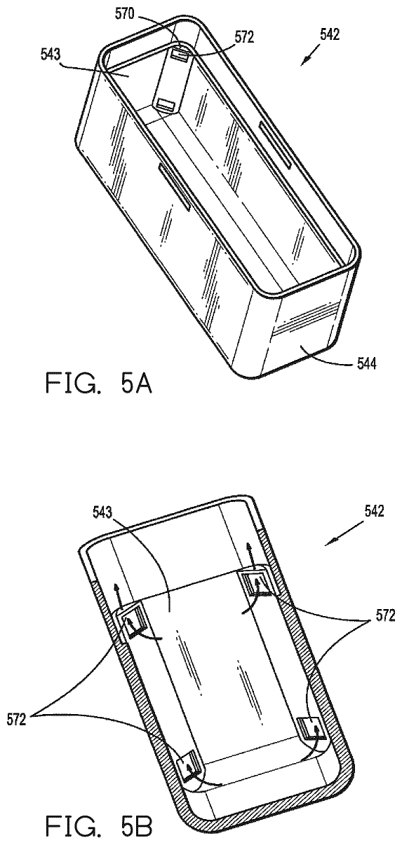

FIGS. 5A and 5B depict another embodiment according to the present disclosure. In FIG. 5A, canister 542 is shown without a canister interface. FIG. 5B is a cross-sectional view of canister 542 shown in FIG. 5A. As shown in FIG. 5B, canister 542 has a left wall 543 with four filters generally shown as 572 located substantially at the comers of the left wall 543. Each filter 572 may have one or more filters that may be hydrophobic or oleophobic. Filter 572 leads to a passageway 570 as shown in FIG. 5A. Although FIG. SB depicts the left wall having four filters, right wall 544 also has similar filters at its comers. As such, the canister 542 has filters located at the eight vertices of the canister 542. Passageway 570 leads to a canister interface (not shown) which has a suction port (not shown) leading to the suction pump (not shown). It is to be appreciated by one skilled in the art that the canister interface for canister 542 may be substantially similar to the canister interface 250 shown in FIG. 4. Unless the canister 542 is completely full of said exudate, one or more of filters 572 located at the eight vertices will not be covered by exudate even when the canister 542 is tipped, tilted, or inverted. Canister 542 allows the canister assembly 240 to be tipped or tilted in any direction while maintaining a negative or reduced pressure at the wound “w”.

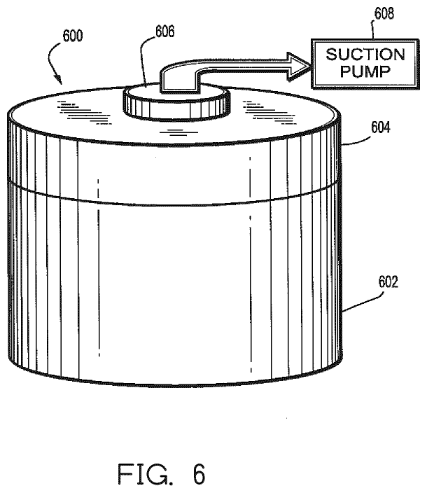

FIG. 6 depicts another embodiment according to the present disclosure. As shown in FIG. 6, a substantially round canister assembly 600 is shown. The base of canister assembly 600 may be circular, oval or elliptical in shape. Canister assembly has a canister 602 a canister interface 604 and a suction port 606. Air is drawn through the suction port 606 by suction pump 608 thereby imparting a negative or reduced pressure in canister 602.

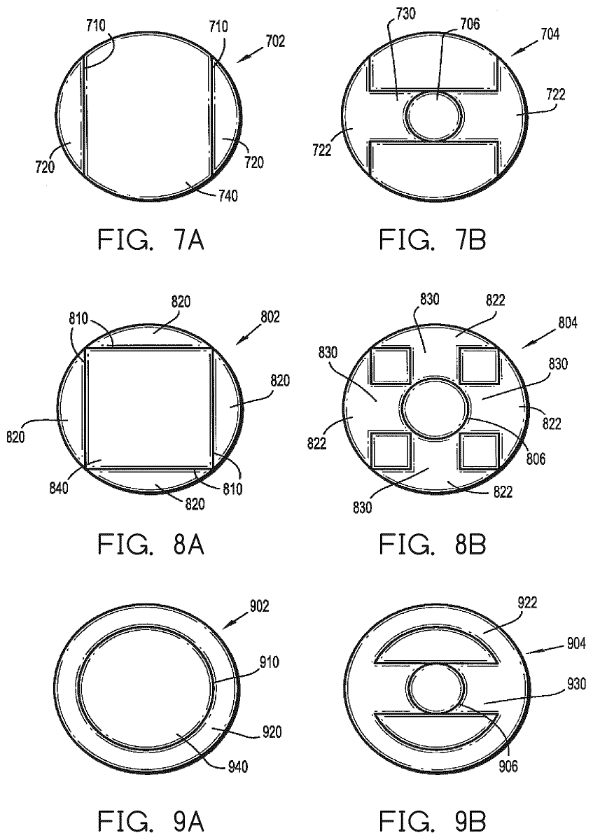

FIGS. 7B and 7B depict top views of the canister and canister interface of FIG. 6 in accordance with an embodiment of the present disclosure. As shown in FIG. 7A, canister 702 has two filters 710, which are substantially similar to the filters described above, that are parallel to each other. Each filter 710 leads to a passageway 720 which leads to the canister interface 704 shown in FIG. 7B. Canister interface 704 has channels 722 which are positioned over passageways 720 of canister 702. Channels 722 lead to central channel 730 which leads to suction port 706. Suction pump 608 (as shown in FIG. 6) draws air through suction port 706 which then draws air through central channel 730. Air is drawn through central channel 730 from channels 722 and passageways 720. Such action causes air to be drawn through filters 710 thereby imparting a negative pressure in chamber 740 of canister 702.

FIGS. 8A and 8B depict top views of the canister and canister interface of FIG. 6 in accordance with an embodiment of the present disclosure. As shown in FIG. 8A, canister 802 has filter assembly 810 that are substantially similar to the filters described above. Filter assembly 810 has two pairs of filters with one pair of filters being perpendicular to the other pair of filters as shown in FIG. 8A. Each filter in filter assembly 810 leads to a passageway 820 which leads to the canister interface 804 shown in FIG. 8B. Canister interface 804 has four channels 822 which are positioned over passageways 820 of canister 802. Channels 822 lead to central channel 830 which leads to suction port 806. Suction pump 608 (as shown in FIG. 6) draws air through suction port 806 which then draws air through central channel 830. Air is drawn through central channel 830 from channels 822 and passageways 820. Such action causes air to be drawn through filter assembly 810 thereby imparting a negative pressure in chamber 840 of canister 802.

FIGS. 9A and 9B depict top views of the canister and canister interface of FIG. 6 in accordance with an embodiment of the present disclosure. As shown in FIG. 9A, canister 902 has a filter assembly 910 that is substantially similar to the filters described above. Filter assembly 910 has a substantially similar shape as the canister. Filter assembly 910 leads to a passageway 920 which leads to the canister interface 904 shown in FIG. 9B. Canister interface 904 has channel 922 which is positioned over passageway 920 of canister 902. Channels 922 lead to central channel 930 which leads to suction port 906. Suction pump 608 (as shown in FIG. 6) draws air through suction port 906 which then draws air through central channel 930. Air is drawn through central channel 930 from channel 922 and passageway 920. Such action causes air to be drawn through filter assembly 910 thereby imparting a negative pressure in chamber 940 of canister 902.

Sensor with Electrical Contact Protection for Use in Fluid Collection Canister and Negative Pressure Wound Therapy Systems Including Same

A sensor for use in a canister for fluid collection, the canister having a canister top and defining a fluid collection chamber. The sensor includes a first electrode and a second electrode. The first electrode includes a first portion and a second portion, wherein the first portion of the first electrode is supported by the canister top, and the second portion of the first electrode is configured to extend into the fluid collection chamber. The second electrode includes a first portion and a second portion, wherein the first portion of the second electrode is supported by the canister top, and the second portion of the second electrode is configured to extend into the fluid collection chamber. The sensor also includes an electric circuit configured to detect an electrical property associated with the first and second electrodes.

Referring to FIG. 10, an embodiment of the canister assembly 240 illustrated in FIG. 2 is shown and includes a control unit 246 and a collection canister 242. In embodiments, canister assembly 240 is coupled via an extension assembly 230 to a dressing assembly (e.g., wound dressing 12 shown in FIG. 1) to apply negative pressure to a wound to facilitate healing of the wound in accordance with various treatment modalities.

Control unit 246 includes a suction pump 1360, a pump inlet conduit 372, a pump outlet conduit 1362, a first filter element 1376, a transducer 1340, a transducer conduit 1352, a second filter element 1354, a first connecting channel 1378 and a second connecting channel 1356.

Suction pump 1360 may provide negative pressure produced by a piston drawn through a cylinder. Suction pump 1360 may be a peristaltic pump or a diaphragm pump. Suction pump 1360 may be a manual pump or an automated pump. The automated pump may be in the form of a portable pump, e.g., a small or miniature pump that maintains or draws adequate and therapeutic vacuum levels. In one embodiment, the suction pump 1360 is a portable, lightweight, battery-operated, DC motor-driven pump. A vibration damping tape (not shown), e.g., visco-elastic damping tape, may be applied to the outer surface of the suction pump 1360 to reduce vibration and its associated noise. Suction pump 1360 may be contained within its own sub-housing (not shown), which may be formed substantially entirely of molded foam, e.g., used as a silencer to provide sound mitigation by reducing the sound energy of the expelled air during operation of the suction pump 1360, and may include a carbon loaded foam. Suction pump 1360 provides negative pressure within the chamber 1335 of the collection canister 242 by drawing air through the suction port 1374.

Pump inlet conduit 1372 provides fluid communication between the suction pump 1360 and the first filter element 1376. The first filter element 1376 may include one or more filters and is configured to substantially prevent entry of exudate into the suction pump 1360. In embodiments, the control unit 246 stops operation of the suction pump 1360 when the first filter element 1376 becomes occluded. A variety of filters can be used for the first filter element 1376. In one embodiment, the first filter element 1376 includes a hydrophobic filter that substantially prevents fluids from entering into the suction pump 1360 and potentially causing damage to electronics or pneumatic components. Exhaust air from the pump 1360 is vented through an exhaust port (not shown) via the pump outlet conduit 1362. Pump outlet conduit 1362 may be coupled to one or more filters (not shown) for filtering the exhaust air from the pump 1360.

Transducer 1340 is in fluid communication with the collection canister 242 to detect the vacuum level within the collection canister 242. In embodiments, the transducer 1340 generates an electrical signal that varies as a function of vacuum level within the collection canister 242, and the signal is communicated to the processor 1310. Logic associated with the transducer 1340 and the pump 1360 may reduce the speed of the pump 1360 or stop operation of the pump 1360 in response to the vacuum level detected by the transducer 1340. Any suitable device capable of detecting pressure may be utilized for the transducer 1340, including, but not limited to, a pressure switch, transducer or transmitter. Transducer conduit 1352 provides fluid communication between the transducer 1340 and the second filter element 1354. In one embodiment, the second filter element 1354 is a hydrophobic filter that substantially prevents fluid contamination of the transducer 1340.

First connecting channel 1378 provides fluid communication between the first filter element 1376 and the suction port 1374, when the control unit 246 and the collection canister 242 are operablely coupled to each other. Second connecting channel 1356 provides fluid communication between the second filter element 1354 and the transducer port 1396, when the control unit 246 and the collection canister 242 are operably coupled to each other. First connecting channel 1378 may be coupled to a control suction port (not shown) located on the bottom side of the control unit 246 and configured to engage with the suction port 1374 located on the collection canister top 1336 when the control unit 246 and the collection canister 242 are joined together. Second connecting channel 1356 may be coupled to a control unit transducer port (not shown) located on the bottom side of the control unit 246 and configured to engage with the transducer port 1396 located on the collection canister top 1336 when the control unit 246 and the collection canister 242 are joined together.

Control unit 246 also includes a processor 1310. In embodiments, the processor 1310 is electrically coupled via a transmission line 1341 to the transducer 1340 and electrically coupled via a transmission line 1361 to the suction pump 1360. Processor 1310 may include any type of computing device, computational circuit, or any type of processor or processing circuit capable of executing a series of instructions that are stored in a memory (not shown) of the control unit 246. The series of instructions may be transmitted via propagated signals for execution by processor 1310 for performing the functions described herein and to achieve a technical effect in accordance with the present disclosure. Control unit 246 may also include a user interface (not shown).

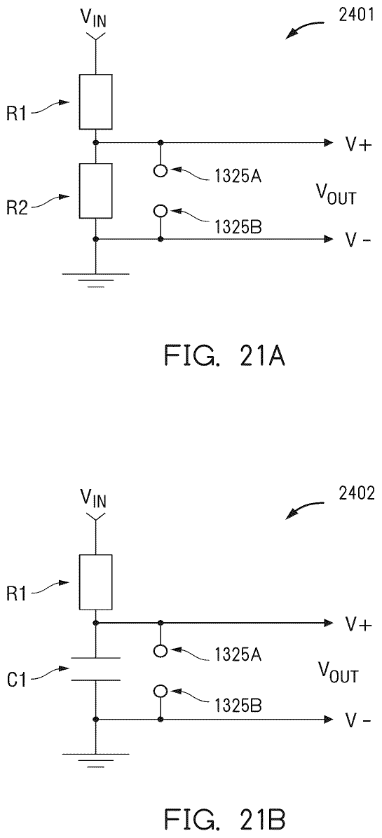

Canister assembly 240 also includes a sensor 1320. Sensor 1320 may include an electrode pair (e.g., 1325A, 1325B shown in FIG. 11). In embodiments, the senor 1320 is used to measure resistance, capacitance or voltage to provide feedback to the processor 1310 indicative of a condition. In embodiments, an electric circuit 1328 is electrically coupled via a transmission line 1321 between the sensor 1320 and the processor 1310. Electric circuit 1328 is configured to detect an electrical property associated with the sensor 1320 and may include various components. Some examples of circuits that may be suitable for use as the electric circuit 1328 are illustrated in the circuit diagrams 2401, 2402 and 2403 shown in FIGS. 21A, 21B and 21C, respectively. Although the electric circuit 1328 is shown as a separate circuit in FIG. 10, it may be incorporated into the sensor 1320, the processor 1310, or other component, e.g., a printed circuit board (not shown) associated with the processor 1310. Sensor 1320 may include multiple electrode pairs (e.g., 1325A, 1325B and 1325C, 1325 1325D shown in FIG. 18). In embodiments, any change in the resistance, capacitance or voltage feedback occurring when the electrodes are simultaneously in contact with exudate in the collection canister 242 is used to indicate a condition, such as, for example, a replace-collection-canister condition or full-collection-canister condition.

Collection canister 242 includes a canister top 1336, a chamber 1335 to collect wound fluids from the dressing assembly, a suction port 1374 to communicate with the chamber 1335 and the suction pump 1360, a canister inlet port 1334 coupled to the extension assembly 230 to introduce the wound fluids from the dressing assembly into the chamber 1335, and a transducer port 1396 to communicate with the chamber 1335 and the transducer 1340. Collection canister 242 may be disposable. Canister inlet port 1334 may be connectable with the extension assembly 230 by conventional air and fluid tight means, such as those described above. In embodiments, canister inlet port 1334 may contain a luer lock or other connector within the purview of those skilled in the art to secure the end of the extension assembly 230 with the canister inlet port 1334. Canister inlet port 1334 may be configured to receive a cap for use to prevent leakage of exudate and odor from the chamber 1335 when the collection canister 242 is separated from the control unit 246.

Referring to FIG. 11, an embodiment of the collection canister 242 of the canister assembly 240 illustrated in FIG. 10 is shown and includes the collection canister top 1336, the chamber 1335, which has length “L1”, the canister inlet port 1334, the suction port 1374, and the transducer port 1396. The sensor 1320 of FIG. 10 includes is shown as an electrode pair 1325A, 1325B in FIG. 11. In embodiments, an electric potential (or voltage) is applied to the electrodes 1325A, 1325B. When a voltage is supplied and the electrodes 1325A, 1325B are simultaneously in contact with an ionic fluid, e.g., exudate, electric current flows via an electro-chemical reaction that occurs between the ions in the fluid and the electrically polarized electrodes 1325A, 1325B.

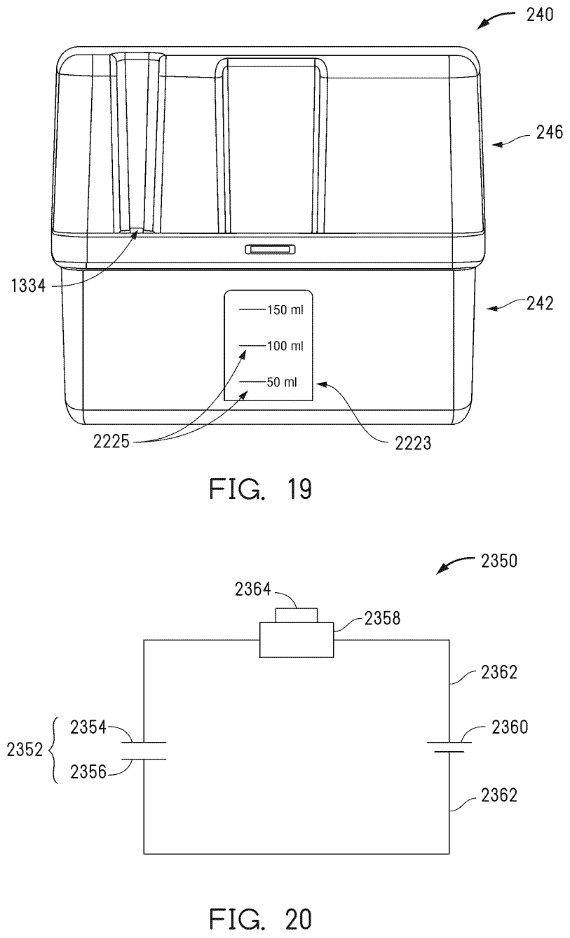

In embodiments, one or more electrode pairs (e.g., 1325A, 1325B shown in FIG. 11) is coupled to an electric circuit (e.g., 1328 shown in FIG. 10), which is configured to detect an electrical properly associated with the electrode pair(s). In embodiments, a measurement of the change in voltage across the electrode pair(s) as a result from the flow of current is used to activate an indicator (e.g., 2358 shown in FIG. 20) as notification to the user of a condition. For example, an indicator may be activated to notify the user that the collection canister 242 is full, which may be referred to as the full-collection-canister condition. An indicator may be activated to notify the user that it is time to replace the collection canister 242, which may be referred to as the replace-collection-canister condition. The occurrence of a replace-collection-canister condition does not necessarily indicate that the chamber 1335 is full of exudate. Rather, a replace-collection-canister condition may indicate that a volume of exudate (less than the volume of the chamber 1335) has been collected. User notification of a replace-collection-canister condition may thus provide some flexibility to the user in the timing of the replacement or empting of the collection canister 242, by allowing an additional time period of operation before the volume of the collected exudate reaches the maximum volume capacity of the chamber 1355.

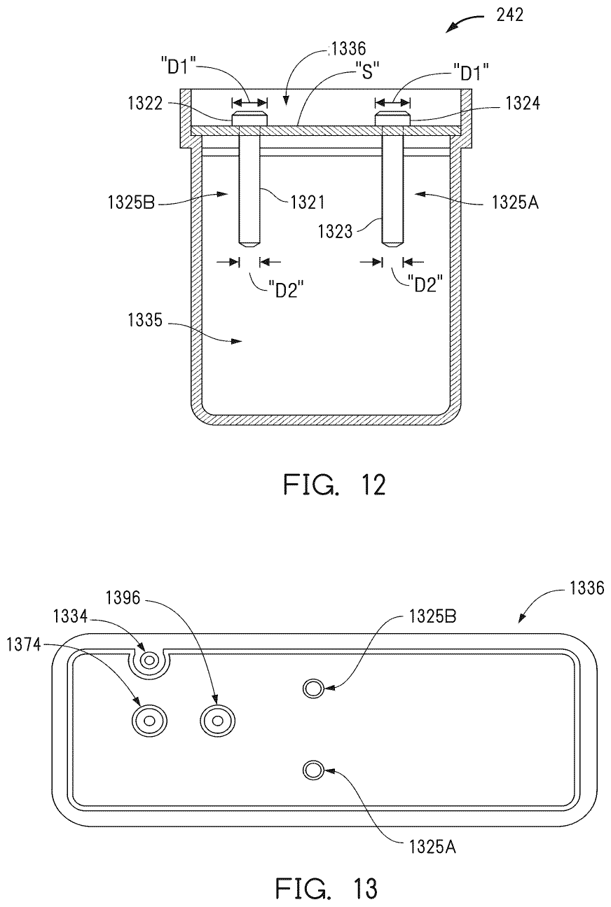

Referring to FIG. 12, a cross-sectional view of the collection canister 242 illustrated in FIG. 11 is shown and includes the collection canister top 1336, the chamber 1335, and the two electrodes 1325A, 1325B. Electrodes 1325A, 1325B include a first portion 1322, 1324 and a second portion 1321, 1323, respectively, wherein the first portions 1322, 1324 have a first diameter “D1” and the second portions 1321, 1323 have a second diameter “D2”, which is smaller than the first diameter “D1”. In an embodiment, the first portions 1322, 1324 are supported by the surface “S” of the collection canister top 1336. In an alternative embodiment, the first portions 1322, 1324 are arranged to be substantially flush with the surface “S” of the collection canister top 1336. Each of the second portions 1321, 1323 extends downwardly from the collection canister top 1336 through a corresponding hole in the collection canister top 1336 and into the chamber 1335. Electrodes 1325A, 1325B, or portions thereof, include electrically conductive material, e.g., metal or metal alloy. Electrodes 1325A, 1325B, or portions thereof, may include an electrically conductive coating. For example, electrodes 1325A, 1325B, or portions thereof, may be plated with an electrically conductive metallic layer. The relative positions, size and/or shape of the two electrodes 1325A, 1325B may be varied from an embodiment depicted in FIG. 12.

In embodiments, two probe contacts (not shown) are located at the bottom side of the control unit 246 and positioned to make electrical contact with the first portions 1322, 1324 of the electrodes 1325A, 1325B, respectively, when the collection canister 242 and the control unit 246 are connected to each other.

FIG. 13 is a bottom view of the collection canister top 1336 illustrated in FIG. 11 shown with the canister inlet port 1334, the suction port 1374, the transducer port 1396 and the two electrodes 1325A, 1325B. The relative positions, size and/or shape of the canister inlet port 1334, the suction port 1374 and the transducer port 1396 may be varied from an embodiment depicted in FIGS. 11 and 13.

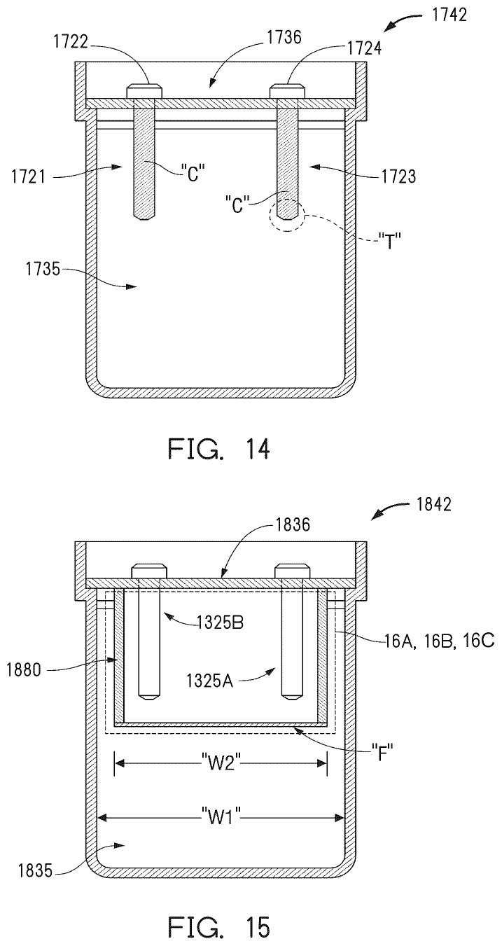

Referring to FIG. 14, an embodiment of a collection canister 1742 is shown and includes a collection canister top 736 and a chamber 1735. Collection canister 1742 is similar to the collection canister 242 illustrated in FIGS. 11 and 12. Collection canister 1742 includes two electrodes with a first portion 1722, 1724 and a second portion 1721, 1723, respectively, which are similar to the two electrodes 1325A, 1325B shown in FIG. 11, except that the second portions 1721, 1723 of FIG. 14 are substantially covered by a coating “C”. In alternative embodiments, coating “C” covers a section of the second portions 1721, 1723, such as the tip section “T”.

In embodiments, the coating “C” is a water-soluble coating for the protection of the second portions 1721, 1723 and may have a predetermined amount of time in contact with fluid before the coating dissolves. Coating “C” may inhibit encrustation of the second portions 1721, 1723 formed by the drying of exudate. For example, the coating “C” may inhibit encrustation by affecting the formation and binding of proteins or may decrease wetability of the second portions 1721, 1723 and allow liquid to shed.

Coating “C” may help to minimize the amount of time that fluid in the chamber 1735 of the collection canister 1742 comes into contact with the second portions 1721, 1723, e.g., during accidental tip over of the canister assembly (e.g., 240 shown in FIG. 19) or during ambulation while the canister assembly is worn or carried by the user. In embodiments, the coating “C” is characterized by a predetermined amount of time in contact with fluid before the coating “C” dissolves. The predetermined amount of time may be a short period of time, e.g., about one hour to about ten hours, which may be suitable in the case of a high rate of flow of exudate from the wound into the chamber 1735. The predetermined amount of time may be a long period of time, e.g., about forty-eight hours, which may be suitable in the case of a low rate of flow of exudate into the chamber 1735. The predetermined amount of time in contact with fluid before the coating “C” dissolves may be varied from the above-mentioned time periods. In some cases, depending on characteristics of the fluid to be collected in the collection canister 1742, the coating “C” may be configured to dissolve at a predetermined rate, e.g., based on the thickness, density and/or composition of the coating “C”.

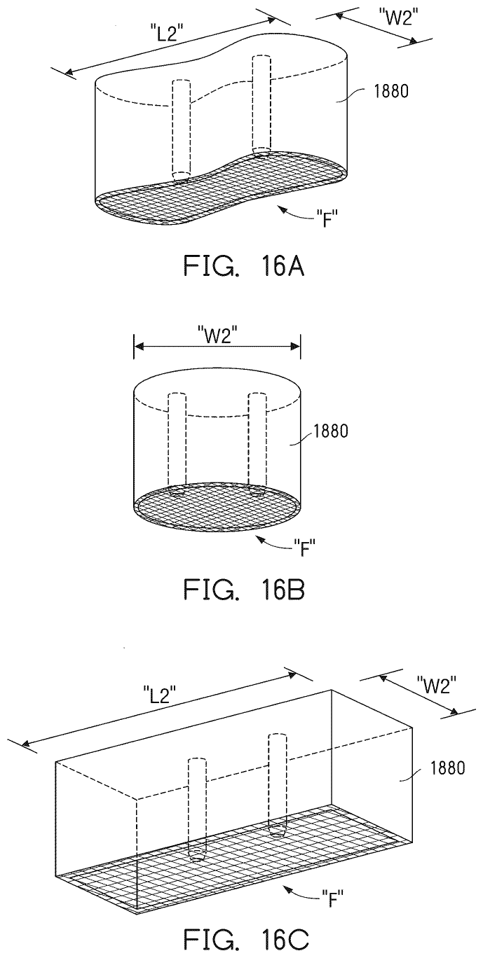

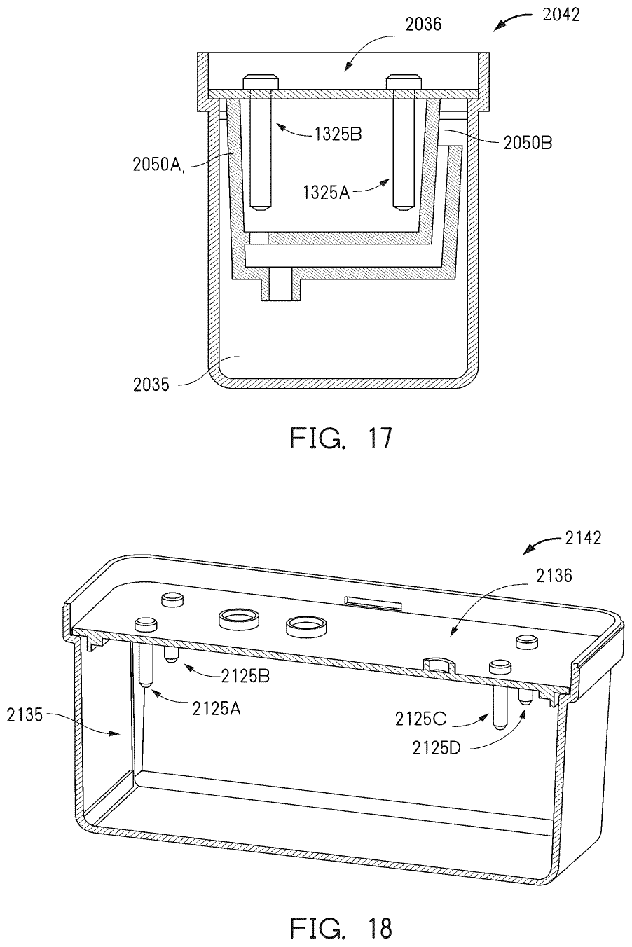

Referring to FIG. 15, an embodiment of a collection canister 1842 is shown and includes a collection canister top 1836 and a fluid collection chamber 1835 having a width “W1”. Collection canister 1842 also includes the two electrodes 1325A, 1325B shown in FIGS. 11 and 12. Collection canister 1842 is similar to the collection canister 242 illustrated in FIGS. 11 and 12, except that the two electrodes 1325A, 1325B are disposed within an electrical contact protection unit bounded by the collection canister top 1836, an inner wall 1880 and a bottom end “F”. The electrical contact protection unit may be formed in various sizes and shapes, such as, for example, the shapes shown in FIGS. 16A, 16B and 16C.

In embodiments, the bottom end “F”, or portion thereof, is formed of a water-soluble film, which is configured to dissolve over a period of time. In embodiments, the water soluble film dissolves over a period of time depending on characteristics of the water soluble film, e.g., thickness, density and/or composition of the water soluble film, characteristics of the fluid contained in the fluid collection chamber, e.g., pH, viscosity and/or ionic composition of the fluid, and/or the rate of flow of the fluid into the fluid collection chamber 1835.

Referring to FIGS. 16A through 16C, embodiments of the electrical contact protection unit of the collection canister 1842 illustrated in FIG. 15 are shown with varied geometric shapes. Each of the electrical contact protection units shown in FIGS. 16A through 16C includes an inner wall 1880 and a bottom end “F”, and each has a width (or maximum width) “W2” that is less than the width “W1” of the fluid collection chamber 1835 shown in FIG. 15. The electrical contact protection units shown in FIGS. 16A and 16C have a length “L2”, which is less than the length “L1” of the chamber 1335 of the canister 242 shown in FIGS. 11 and 12. The relative positions, size and/or shape of the electrical contact protection units may be varied from embodiments depicted in FIGS. 15 through 16C.

Referring to FIG. 17, an embodiment of a collection canister 2042 is shown and includes a collection canister top 2036 and a chamber 2035. Collection canister 1842 also includes the two electrodes 1325A, 1325B shown in FIGS. 11 and 12. Collection canister 1842 is similar to the collection canister 242 illustrated in FIGS. 11 and 12, except that the two electrodes 1325A, 1325B are disposed within a baffle unit formed of two baffle members 2050A, 2050B. Baffle members 2050A, 2050B may be formed in various sizes and shapes.

Referring to FIG. 18, an embodiment of a collection canister 2142 is shown and includes a collection canister top 2136, a chamber 2135, a first electrode pair 2125A, 2125B and a second electrode pair 2125C, 2125D. In one embodiment, a measurement of the change in voltage across the first electrode pair 2125A, 2125B is used to activate an indicator as notification to the user of a replace-collection-canister condition, and a measurement of the change in voltage across the second electrode pair 2125C, 2125D is used to activate an indicator as notification to the user of a full collection canister condition.

Referring to FIG. 19, the canister assembly 240 of the negative pressure wound therapy system illustrated in FIG. 2 is shown and includes the collection canister 242 shown with a transparent or semi-transparent portion 2223. Transparent or semitransparent portion 2223 includes fluid level markings or graduations 2225 and may help to permit a visual assessment of the amount of exudate contained within the collection canister 242, and, thus, may assist in determining the remaining capacity of the collection canister 242 and/or when the collection canister 242 should be replaced or emptied. Transparent or semi-transparent portion 2223 may help to permit a visual assessment of the wound exudate to assist in evaluating the color, quality and/or quantity of exudate.

Referring to FIG. 20, an embodiment of a detector circuit 2350 is shown and includes a sensor 2352, incorporating electrically conductive contacts 2354 and 2356, and indicator unit 2358. Contacts 2354 and 2356 may be disposed within the collection canister 242 at a pre-determined height or level within the collection canister 242 corresponding to a targeted volume of fluid or exudate accumulated within the collection canister 242. For example, contacts 2354 and 2356 may be positioned within the collection canister 242 at a position corresponding to the maximum fill volume of collection canister 242.

As illustrated in FIG. 20, contacts 2354 and 2356 of sensor 2352 are laterally spaced. Contacts 2354 and 2356 are connected via transmission lines 2362 to the indicator unit 2352 and power source 2360. Alkaline batteries, wet cell batteries, dry cell batteries, nickel cadmium batteries, lithium batteries, NiMH batteries (nickel metal hydride), solar energy and other energy sources may serve as the power 2360. Power source 2360 may be a separate unit from the power source utilized to energize the vacuum source (e.g., 1360 shown in FIG. 10).

In embodiments, the indicator unit 2358 of the detector circuit 2350 generates an alert or signal that the canister 242 is full of a fluid, e.g., exudate. Indicator unit 2358 may be any type of indicator capable of alerting the user or clinician that the canister 242 needs to be replaced. Indicator unit 2358 may be an audio and/or visual indicator. In an embodiment, the indicator unit 2358 includes an alarm or output component 2364, which includes logic or circuitry to generate a signal when power is provided to the indicator unit 2358. Output component 164 is adapted to provide a perceptible sensory alert, which may be an audio, visual, or other sensory alarm. In one embodiment, the indicator 2358 is adapted to generate an audio signal and the output component 2364 includes an audio circuit with a speaker. In one embodiment, the indicator 2358 is adapted to generate a visual signal and the output component 2364 includes a light source, such as a light-emitting diode (LED).

FIGS. 21A and 21B are schematic diagrams of detection circuits 2401, 2402, respectively, for use to detect a voltage measurement across an electrode pair, e.g., the electrodes 1325A, 1325B shown in FIGS. 11 and 12. Detection circuit 2401 includes a first resistor R1 and a second resistor R2. In an embodiment, wherein the first resistor R1 is a 10000000 ohm (10000K or 10M) resistor, the second resistor R2 is a 24M ohm resistor, and V_{IN} equals 3.3V, the detection circuit 2401 may produce a constant 2.4V output (VouT) when no exudate has shorted the two electrodes 1325A, 1325B.

Referring to FIG. 21B, the detection circuit 2402 includes a resistor R1 and a capacitor C1. In an embodiment, wherein the resistor R1 is a 100000 ohm (100K) resistor, the capacitor C1 is a 100 pf capacitor, and V_{IN} equals 3.3V, the detection circuit 2402 may produce a constant 3.3V output (Vou T) when no exudate has shorted the two electrodes 1325A, 1325B.

In the detection circuits 2401 and 2402, when the two electrodes 1325A, 1325B make contact with an ionic fluid, e.g., exudate, electric current flows via an electrochemical reaction that occurs between the ions in the fluid and the electrically polarized electrodes 1325A, 1325B, resulting in a change in the measured voltage output (VouT). In embodiments, a measurement of the change in voltage across the electrode pair(s) as a result from the flow of current is used to activate an indicator (e.g., 2358 shown in FIG. 20) as notification to the user to replace or empty the collection canister (e.g., 242 shown in FIGS. 11 and 12).

Canister for Receiving Wound Exudate in a Negative Pressure Therapy System

A system to promote the healing of an exuding wound includes a wound dressing, a subatmospheric pressure mechanism, and a collection canister. The wound dressing is dimensioned for positioning relative to a wound bed of a subject. The subatmospheric pressure mechanism includes a control unit disposed within a housing. The control unit includes a vacuum source associated with a vacuum port. The collection canister has an interior wall which defines an internal chamber. The internal chamber is in fluid communication with the vacuum source of the subatmospheric pressure mechanism through the vacuum port and with the wound dressing for collecting exudates removed for the wound bed under influence of the vacuum source. The canister including a baffle mechanism disposed within the internal chamber for dampening the movement of the collected exudates.

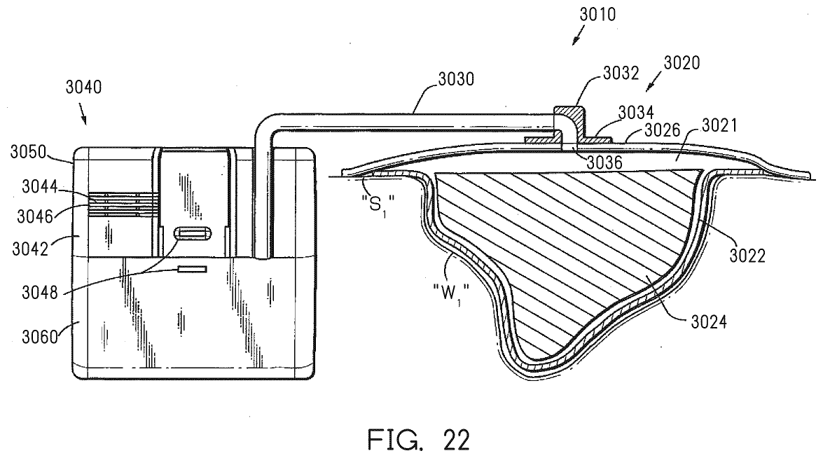

Referring initially to FIG. 22, the wound therapy system 3010 according to the present disclosure is illustrated for use on a wound “w₁” surrounded by healthy skin “si.” Wound therapy system 3010 includes composite wound dressing 3020, subatmospheric pressure mechanism 3040, and collection canister 3060 in fluid communication with the wound dressing 3020 through conduit 3030.

Wound dressing 3020 is positioned relative to the wound “w₁” to define a reservoir 3021 in which a negative pressure appropriate to stimulate healing may be maintained. Wound dressing 3020 may include several components, namely, wound contact layer or member 3022, a wound packing member or filler 3024 supported by the contact member 3022 and outer layer or cover member 3026. Wound contact member 3022 is adapted to substantially conform to the topography of a wound bed “w₁.” Wound contact member 3022 may be substantially porous or perforated to permit exudates to pass from the wound bed “w₁” through the wound contact member 3022. The passage of wound exudates through the wound contact member 3022 may be unidirectional such that wound exudates do not flow back to the wound bed “w₁.” Unidirectional flow may be encouraged by directional apertures formed in contact member 108, lamination of materials having absorption properties differing from those of contact member 3022, or by selection of materials that promote directional flow. A non-adherent material may be selected such that contact member 3022 does not tend to cling to wound bed “w₁” or surrounding material when it is removed. Exemplary materials that may be used as contact member 3022 are sold under the trademarks XEROFORM® and CURITY®, offered by Tyco Healthcare Group LP (d/b/a Covidien).

Wound packing member 3024 of wound dressing 3020 is intended to transfer wound fluid and exudates. Wound packing member 3024 is conformable to assume the shape of any wound bed “w₁” and may be packed up to the level of healthy skin “si.” Wound packing member 3024 may be pre-formed in any shape and size or may be custom fit by cutting the packing member 3024 to a desired shape and/or size. Wound packing member 3024 may be treated with agents to promote healing of the wound, such as polyhexamethylene biguanide (PHMB) to decrease the incidence of infection, or other substances having clinical use, such as other medicaments. Suitable materials for wound packing member 3024 are sold under the trademarks KERLIX®, EXCILON®, and WEBRIL®, all by Tyco Healthcare Group LP (d/b/a Covidien).

Outer member or wound covering 3026 encompasses the perimeter of the wound dressing 3020 to surround wound bed “w₁” and to provide a liquid and/or fluid tight seal around the perimeter “pi” of the wound bed “w₁.” For instance, the sealing mechanism may be any adhesive bonded to the perimeter of wound covering 3026. One exemplary material that may be used as an adhesive dressing is sold under the trademark CURAFORM® Island Dressing by Tyco Healthcare Group LP (d/b/a Covidien). Thus, wound covering 3026 may act as both a microbial barrier and a fluid barrier to prevent contaminants from entering wound bed “w₁” and for maintaining the integrity thereof.

Wound covering 3026 is typically a flexible material, e.g., resilient or elastomeric, that seals the top of wound dressing 3020 to prevent passage of liquids, fluids, and/or contaminants to and from the wound dressing 3020. Wound covering 3026 may be formed from a moisture vapor permeable membrane to promote the exchange of oxygen moisture between the wound bed “w₁” and atmosphere. A membrane that provides a sufficient moisture vapor transmission rate is a transparent membrane sold under the trademark POLYSKIN® II by Tyco Healthcare Group LP (d/b/a Covidien). A transparent membrane permits an assessment of wound conditions without requiring removal of the wound covering 3026. Alternatively, wound covering 3026 may comprise an impermeable membrane or a substantially rigid membrane.

Wound covering 3026 may include a port or connector 3032 in fluid communication with the interior of wound dressing 3020 to facilitate connection of wound dressing 3020 to conduit or tubing 3030. Conduit 3030 defines a fluid flow path leading through wound therapy system 3010. Connector 3032 may be configured as a rigid or flexible, low-profile component, and may be adapted to receive conduit 3030 in a releasable and fluid tight manner. An adhesive on the underside of flange 3034 of connector 3032 may provide a mechanism for affixing the conduit 3030 to the wound dressing 3020 or alternatively, flange 3034 may be positioned within reservoir 3021 such that an adhesive on an upper side of the flange 3034 affixes the conduit 3030. However it is affixed to wound dressing 3020, a hollow interior 3036 of connector 3032 provides fluid communication between conduit 3030 and the interior of wound dressing 3020, such as reservoir 3021.

Connector 3032 may have a valve (not shown) built therein or in line with conduit 3030, e.g., a one-way valve to permit exudates to flow in one direction only, i.e., away from wound dressing 3020 toward subatmospheric pressure mechanism 3040. Connector 3032 may be provided as a pre-affixed component of wound dressing 3020, as a component of conduit 3030 or entirely separate and connected thereto by conventional means. Alternatively, connector 3032 may be eliminated if other provisions are made for providing fluid communication between wound dressing 3020 and conduit 3030.

Conduit 3030 extends from subatmospheric pressure mechanism 3040 to provide fluid communication between the interior of the wound dressing 3020 and subatmospheric pressure mechanism 3040. Any suitable conduit may be used including those fabricated from flexible elastomeric or polymeric materials. Conduit 3030 may connect to subatmospheric pressure mechanism 3040 or other system components by conventional air tight means such as friction fit, bayonet coupling, or barbed connectors. The conduit connections may be made permanent, or alternatively a quick-disconnect or other releasable means may be used to provide some adjustment flexibility to the apparatus.

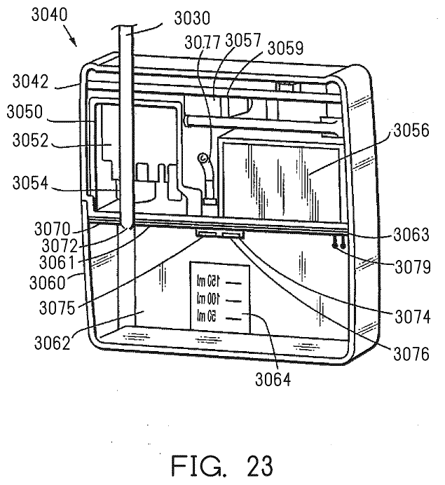

Referring now to FIG. 23, in conjunction with FIG. 22, subatmospheric pressure mechanism 3040 and collection canister 3060 will be discussed. Subatmospheric pressure mechanism 3040 includes housing 3042 and control unit 3050 disposed within the housing 3042. Housing 3042 may be any suitable rigid member which may be adapted for donning by the subject. Control unit 3050 may incorporate vacuum source or pump 3052, actuator or motor 3054 for activating the vacuum source 3052, and power source 3056. Vacuum source or pump 3052 generates or otherwise provides negative pressure to wound therapy system 3010.

Vacuum source or pump 3052 may be a pump of the diaphragmatic, peristaltic or bellows type or the like, in which the moving part(s) draw exudates out of the wound bed “w₁” into the wound dressing 3020 by creating areas or zones of decreased pressure e.g., vacuum zones with the wound dressing 3020 appropriate to stimulate healing of the wound. This area of decreased pressure may communicate with the wound bed “w₁” to facilitate removal of the fluids therefrom and into packing member 3024.

Vacuum source or pump 3052 may be a miniature pump or micropump that may be biocompatible and adapted to maintain or draw adequate and therapeutic vacuum levels. The vacuum level of subatmospheric pressure achieved may be in the range of about mmHg to about 500 mmHg. In embodiments, the vacuum level may be about 75 mmHg and about 125 mmHg, or between about 30 mmHg and 80 mmHg. Vacuum source or pump 3052 is actuated by actuator 3054 which may be any means known by those skilled in the art, including, for example, AC motors, DC motors, voice coil actuators, solenoids, and the like. Actuator 3054 may be incorporated within pump 3052.

Power source 3056 may be disposed within housing 3042 or separately mountable to housing 3042. A suitable power source 3056 includes alkaline batteries, wet cell batteries, dry cell batteries, nickel cadmium batteries, solar generated means, lithium batteries, NiMH batteries (nickel metal hydride) each of which may be of the disposable or rechargeable variety.

Subatmospheric pressure mechanism 3040 may also include a pressure transducer 3057 which may be attached to a printed circuit board (PCB) 3059. Within the PCB 3059 is software or circuitry that powers the pressure transducer 3057 and receives its pressure signals (i.e., electrical signals indicative of the negative pressure being measured).

Housing 3042 may include vent portal 3044 configured to vent exhaust air from vacuum source or pump 3052 through an exhaust port (not shown). Vent portal 3044 extends from housing 3042 and may be directly connected to vacuum source 3052. It is also envisioned that vent portal 3044 may exhaust air from within housing 3042 rather than directly from vacuum source 3052. A filter 3046 may extend across vent portal 3044. Filter 3046 may be a bacterial and/or odor control filter to prevent emission of bacteria and/or odors from housing 3042.

Collection canister 3060 collects exudates “e” removed from the wound bed “w₁” during therapy through conduit or tubing 3030. Collection canister 3060 is associated with housing 3042 and may be incorporated within the housing 3042 or releasably connected to the housing 3042 by conventional means. Housing 3042 and collection canister 3060 may be releasably coupled via mating members 3048. Mechanisms for selective coupling and decoupling of housing 3042 and collection canister 3060 include fasteners, latches, clips, straps, bayonet mounts, magnetic couplings, and other devices for selective mating of housing 3042 and collection canister 3060.

Collection canister 3060 may comprise any container suitable for containing wound fluids and is substantially rigid defining an internal chamber 3062 in fluid communication with tubing 3030. In the alternative, collection canister 3060 may be relatively flexible. In embodiments, at least a portion of collection canister 3060 may be transparent to assist in evaluating the color, quality, or quantity of wound exudates. A transparent or partially transparent window 3064 may thus assist in determining the remaining capacity of the canister 3060 or when the canister 3060 should be replaced.

Collection canister 3060 includes fluid inlet 3072 and suction port 3074. Collection canister 3060 may also include pressure transducer port 3075 and/or fill sensor 3079. Fluid inlet 3072 is configured to operably engage conduit 3030. Fluid inlet 3072 may be connectable with conduit 3030 by conventional air and fluid tight means, such as those described above and terminates within internal chamber 3062 to deposit fluids and exudates conveyed by conduit 3030 into internal chamber 3062. In embodiments, fluid inlet 3072 may contain a luer lock or other connector within the purview of those skilled in the art to secure the end of conduit 3030 with the fluid inlet 3072. It is envisioned that fluid inlet 3072 is configured to receive a cap or a tubing clamp in order to prevent leakage of exudates and odor from internal chamber 3062 of collection canister 3060 when housing 3042 is separated from the canister 3060.

Suction port 3074 of collection canister 3060 is in fluid communication with vacuum source or pump 3052 of subatmospheric pressure mechanism 3040. Pump 3052 creates a vacuum within internal chamber 3062 of collection canister 3060 by drawing air through suction port 3074. A filter 3076, such as a hydrophobic membrane or baffling to prevent exudates from being aspirated into pump 3052, may be disposed within suction port 3074. Alternatively, filter 3076 may be disposed adjacent to suction port 3074 such that suction port 3074 passes air between vacuum source 3052 and canister 3060 through filter 3076 while keeping the contents from reaching the vacuum pump 3052 or other components of control unit 3050.

The hydrophilic nature of the filter 3076 allows the collection canister 3060 to be oriented in a way other than with the pump 3052 above the canister 3060, such as on the side of the canister 3060 or tipped, without exudates in the canister 3060 being aspirated into pump 3052. Some portion of the surface of filter 3076 remains uncovered, thereby allowing continued flow of air to vacuum pump 3052. Filter 3076 may include charcoal or other odor absorbing materials and may prevent the passage of bacteria.

Pressure transducer port 3075 is in fluid communication with pressure transducer 3057 through tube 3077 and permits the monitoring of pressure levels within internal chamber 3062 of collection canister 3060. Filter 3076 may be disposed between pressure transducer port 3075 and internal chamber 3062 of collection canister 3060 to prevent the migration of fluids or exudates into the pressure transducer port 3075 and the pressure transducer 3057.

Fill sensor 3079 is disposed within collection canister 3060 at a pre-determined height or level within the canister 3060 corresponding to a targeted volume of fluid or exudates accumulation within the canister 3060, such as at a position corresponding to a maximum fill volume of the canister 3060. Fill sensor 3079 incorporates electrically conductive contacts which are connected to an indicator which generates an alert or signal that the canister 3060 is full.

In embodiments, collection canister 3060 includes canister insert 3061. Canister insert 3061 is dimensioned to fill the opening of canister 3060 and to be placed within internal chamber 3062 of canister 3060 until it engages a lip 3063 around at least a portion of the peripheral inner edge of canister 3060 or frictionally engages the inner walls of the canister in a fluid tight, yet releasable manner. Canister insert 3061 may include the fluid inlet 3072, suction port 3074, pressure transducer port 3075, and/or filter 3076. Alternatively, the components discussed above may be maintained within a top cover portion of collection canister 3060 or may be maintained within a lower bottom portion of the housing 3042 of subatmospheric pressure mechanism 3040.

Collection canister 3060 includes baffle mechanism 3100 disposed within internal chamber 3062 of the canister 3060. Baffle mechanism 3100 includes components for dampening exudates movement which may occur from user movement, such as bending or otherwise reorienting the body, or through waves of “sloshing” caused by cyclical movements, such as walking. The baffle mechanism 3100 is effective against fluid movement without affecting the collection capability of canister 3060.

The baffle mechanism 3100 may include three dimensional structures or baffles 3110 disposed freely within the canister 3060 or formed as part of, or attached to, the collection canister 3060. The baffle mechanism 3100 provides additional surface area within collection canister 3060 thereby dividing the surface area of the exudates within the canister 3060 into smaller areas. The additional surfaces resist motion, such as wave action or liquid sloshing, of the exudates. The additional surfaces also serve to dampen the movement of the exudates in the canister thereby at least partially absorbing the kinetic energy of the exudates.

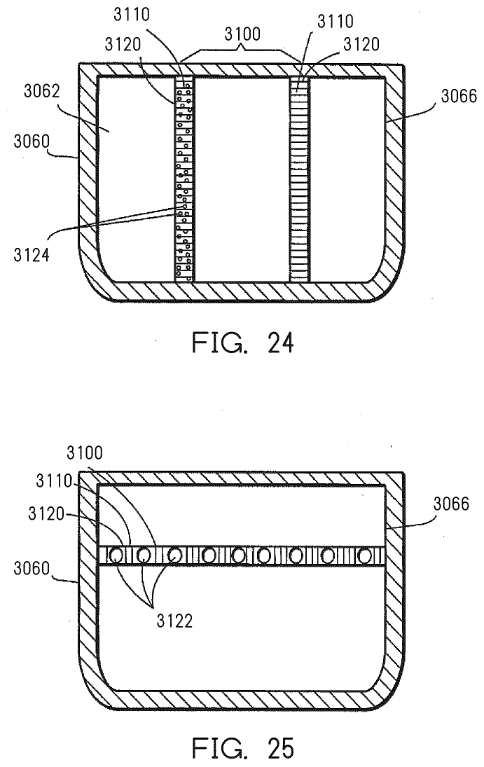

In an embodiment of the baffle mechanism of the present disclosure depicted in FIGS. 24-26, baffle mechanism 3100 includes at least one baffle 3110 fabricated from a material segment 3120. Material segment 3120 may be formed from woven or nonwoven materials in the form of a knit, weave, mesh, sheet, web, or other construct within the purview of those skilled in the art that has been formed by mechanically, chemically, or thermally bonding of the material. The nonwoven material may have a structure of fibers or threads which are interlaid or bonded in a random or systematic manner through a variety of processes, such as, for example, melt-blowing or spunbonding. The fibers may be natural or man-made fibers, such as regenerated or synthetic fibers. Exemplary materials include, but are not limited to, polypropylene, high and low density polyethylene, ethylene copolymers, propylene copolymers, polyvinyl chloride, polyesters, polyamides, polyfluorocarbons, polyurethane, polystyrene, polyvinyl alcohol, caprolactams, cellulosic and acrylic resins, and bicomponents and/or blends thereof.

Material segment 3120 may also be hydrophobic so that it will not absorb wound exudates as it is collected in canister 3060. Hydrophobic materials include, for example, polyolefins and polyesters, as listed above.

The material segment 3120 may have a low apparent density of less than about 0.1 grams per cubic centimeter. The material segment 3120 may also have a relatively high fiber diameter. In embodiments, the fiber diameter is about 1 micron to about 14 microns. In some embodiments, the fiber diameter is greater than 15 microns.

Material segment 3120 may also be permeable to allow for flow of exudates, gases and/or liquids, therethrough. Material segment 3120 may include apertures 3122, as illustrated in FIG. 25, to facilitate the passage of fluids, such as gases and liquids, therethrough. Material segment 3120 may be disposed only with the upper portion of canister 3060 thereby allowing for relatively free movement of exudates within the canister bottom.

In embodiments, material segment 3120 is treated with an antimicrobial agent 3124. The permeable or porous nature of the material segment 3120 allows exudates to pass through and be treated by the antimicrobial agent 3124. For example, the antimicrobial agent may be imparted to the material segment 3120, or to the individual fibers thereof, by any method within the purview of those skilled in the art, such as by distributing the agent throughout the material or by providing a coating thereon. The antimicrobial agent may then be released from the material segment 3120 upon contact with moisture.

Antimicrobial agents include compounds that kill microorganisms and/or prevent or inhibit their growth or production. Antimicrobials may be antibiotics, antiseptics, anti-fungals, and combinations thereof. The antimicrobial agents utilized within the present disclosure include triclosan, also known as 2,4,4′-trichloro-2′-hydroxydiphenyl ether, the biguanides, especially chlorhexidine and its salts, including chlorhexidine acetate, chlorhexidine gluconate, chlorhexidine digluconate, chlorhexidine hydrochloride, and chlorhexidine sulfate, silver and its salts, including silver acetate, silver benzoate, silver carbonate, silver citrate, silver chloride, silver iodate, silver iodide, silver lactate, silver laurate, silver nitrate, silver oxide, silver palmitate, silver zeolite, silver protein, and silver sulfadiazine, polymyxin, tetracycline, aminoglycosides, such as tobramycin and gentamicin, rifampician, bacitracin, neomycin, chloramphenical, miconazole, tolnaftate, quinolones such as oxolinic acid, norfloxacin, nalidix acid, pefloxacin, enoxacin and ciprofloxacin, penicillins such as ampicillin, amoxicillin, oxacillan, and piracil, cephalosporins, vancomycin, and combinations of any of the above antimicrobials.

In other embodiments, suitable biguanides which may be utilized as antimicrobial agents include hexamethylene biguanides, oligo-hexamethyl biguanides, and/or water-soluble polymers, e.g. polyhexamethylene biguanide (PHMB), or a suitable salt thereof. Any polymeric biguanide within the purview of those skilled in the art may be used herein.

Polymeric biguanides useful herein may include oligo- or poly alkylene biguanides or salts thereof or mixtures thereof. Suitable salts include water-soluble salts with inorganic or organic acids, for example hydrochlorides, hydrobromides, borates, acetates, gluconates, sulfonates, maleates, ascorbates, stearates, tartrates or citrates. Examples of specific polymeric biguanides and salts thereof which may be utilized as antimicrobials include polyhexamethylene biguanide, polyhexamethylene biguanide hydrochloride, polyhexamethylene biguanide hydrobromide, polyhexamethylene biguanide borate, polyhexamethylene biguanide acetate, polyhexamethylene biguanide gluconate, polyhexamethylene biguanide sulfonate, polyhexamethylene biguanide maleate, polyhexamethylene biguanide ascorbate, polyhexamethylene biguanide stearate, polyhexamethylene biguanide tartrate, polyhexamethylene biguanide citrate, and combinations thereof.

In some embodiments, PHMB hydrochloride, PHMB stearate, and the like may be utilized. Polyhexamethylene biguanide hydrochloride is a polymeric material that is commercially available as COSMOCIL® CQ from ARCH® Biocides, a division of Arch Chemicals, Inc. (Norwalk, Conn.). PHMB hydrochloride is active against a wide range of microorganisms, it has very low mammalian toxicity, and it is chemically stable. Polyhexamethylene biguanide hydrochloride is also referred to as polyaminopropyl biguanide by the Cosmetic Toiletries and Fragrances Association (CTFA).

Anti-fungals which may be utilized with the present disclosure include azoles, polyenes, allylamines, and echinocandins. Examples include miconazole, ketoazole, econazole, itraconazole, sertaconazole, fluconazole, voriconazole, clioquinol, bifoconazole, terconazole, butoconazole, tioconazole, oxiconazole, sulconazole, saperconazole, clotrimazole, anidulafungin, caspofungin, micafungin, undecylenic acid, haloprogin, flucytosine, butenafine, tolnaftate, nystatin, ciclopirox, benzoic acid, olamin, terbinafine, amorolfine, naftifine, elubiol, griseofulvin, salts and/or combinations thereof.

Natural antimicrobial agents may also be utilized in the present disclosure. Natural antimicrobial agents include cinnamon oil, cinnamaldehyde, lemongrass oil, clove oil, saw palmetto extract, thyme oil white, thyme oil red, thymol, tea tree oil, pinus pinaster bark extract, rosemary leaf extract, grape seed extract, and betel oil.

In some illustrative embodiments of the present disclosure colorants, emulsifiers, surfactants, and color stabilizers that are well known within the art are added to the material segment 3120. The colorants, in the form of dyes or pigments, aid in reducing shelf life discoloration or discoloration due to the effects of sterilization. The addition of emulsifiers and surfactants aid in surface wettability. Color stabilizers are sometimes added when the antimicrobial is a silver salt.

Material segment 3120 may be formed as part of collection canister 3060, may be an attachment thereto, or may be a removable insert adapted for positioning within collection canister 3060. As such, material segment 3120 may be any shape and size and come in a variety of configurations, such as a straight, pleated, ribboned, or random design. Additionally, one or more of the same or different material may be connected together to form material segment 3120. Further, material segment 3120 may be produced by any number of manufacturing techniques. Material segment 3120 may, for example, be heat formed, cut, stamped, patterned, pressed, extruded, or molded into a desired configuration.

The material segment 3120 may be anchored to opposing sides of interior wall 3066 of canister 3060 as illustrated in FIGS. 24-25, or connected between any two points within canister 3060. Any number of material segments 3120 may be included therein. Material segment 3120 may be attached in any manner, including, gluing, melting, welding, riveting, stapling, and other adhesion methods within the purview of those skilled in the art.

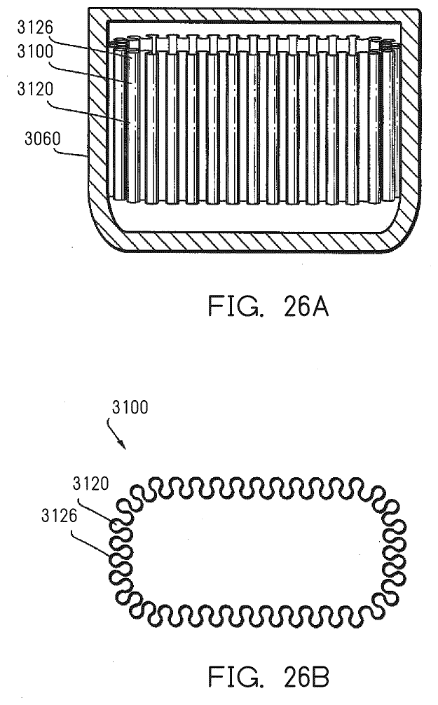

Alternatively, if the material segment 3120 is configured as an insert 3126, as illustrated in FIGS. 26A-26B, the material segment must be of a sufficient rigidity, thickness, or size to maintain its form within canister 3060. Further, as illustrated, the insert 3126 may be shaped to be circumferentially compressed within canister 3060. Alternatively, insert 3126 may be freely disposed within canister 3060 to move with the exudates. Upon movement of canister 3060, kinetic energy of the exudates may be uniformly imparted on the mutually moving insert 3126.

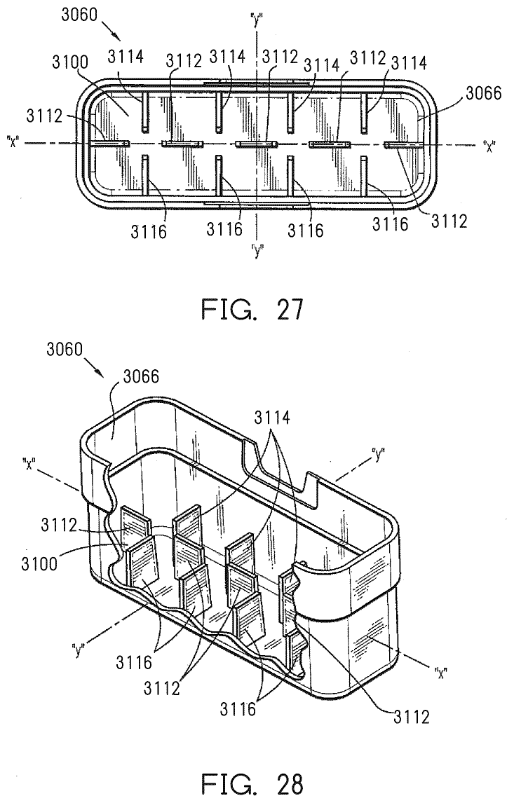

In another embodiment of the baffle mechanism 3100 of the present disclosure as depicted in FIGS. 27-28, the baffle mechanism 3100 includes a plurality of baffles depending from the interior wall 3066 of the collection canister 3060. The baffles may be material segments 3120 as described above which have been treated with antimicrobial agent(s). The baffles may also or alternatively be treated with other bioactive agents, coated with a polymeric material to impart desired physical characteristics such as permeability and flexibility, or combinations thereof. Moreover, the plurality of baffles 3110 may be fabricated from the same or different materials and accordingly, have uniform or varying properties.

The bioactive agent may be any substance or mixture of substances that have clinical use. Consequently, bioactive agents may or may not have pharmacological activity per se, e.g., a dye or fragrance. Alternatively, a bioactive agent could be any agent that provides a therapeutic or prophylactic effect to the collected wound exudates, a compound that affects or participates in tissue growth, cell growth, or cell differentiation, a compound that may be able to invoke a biological action, or could play any other role in one or more biological processes. It is envisioned that the bioactive agent may be treated on the baffle in any suitable form of matter, e.g., powders, gels, or films, by any suitable method for distributing the agent throughout the material or providing a coating thereon, including for example, spraying, dipping, brushing, and melting of the bioagent onto the baffle.

Examples of classes of bioactive agents which may be utilized in accordance with the present disclosure include antimicrobials, antibacterials, antibiotics, anti-virals, and anti-fungals for controlling the growth of microorganisms in the collected wound exudates. The bioactive agent may also be a fragrance, such as an odor control scent powder, for controlling the odor of the exudates. It is also intended that combinations of bioactive agents may be used.

Collection canister 3060 includes longitudinal axis “x” and orthogonal axis “y” which is substantially perpendicular to longitudinal axis “x.” Baffles depend from interior wall 3066 of the canister 3060 and may be positioned in any orientation relative to the longitudinal axis “x” and the orthogonal axis “y.” For example, some or all baffles may be oriented parallel to the longitudinal axis “x,” the orthogonal axis “y,” and/or transverse to both axes.

As illustrated in the current embodiment, canister 3060 includes groups of baffles, each group including similarly or identically oriented baffles within canister 3060. A first group includes one or more baffles 3112 which are oriented in parallel and spaced relation with respect to the longitudinal axis “x”. In embodiments, baffles 3112 are coincident with axis “x.” A second group includes one or more baffles 3114 which are oriented in transverse and spaced relation with respect to the longitudinal axis “x.” Baffles 3114 may be positioned at any angle that is crosswise to the longitudinal axis, in embodiments, the baffles 3114 are perpendicular to the longitudinal axis and are parallel to the orthogonal axis “y.” A third group includes one or more baffles 3116 which are also arranged in space relation with respect to the longitudinal axis. Baffles 3116 may be arranged in a general diametrical opposed relation with respect to baffles 3114.

The groups of baffles 3112, 3114, 3116 define a serpenditious path along at least the lower section of canister 3060 through which the exudates must navigate during tilting and/or maneuvering of the canister 3060, which occurs during ambulatory movement of the subject. In addition, the arrangement of baffles 3112, 3114, 3116 substantially increases the surface area or contact area of the lower section of canister 3060 with the exudate thereby increasing the absorption of the kinetic energy of the moving exudates.

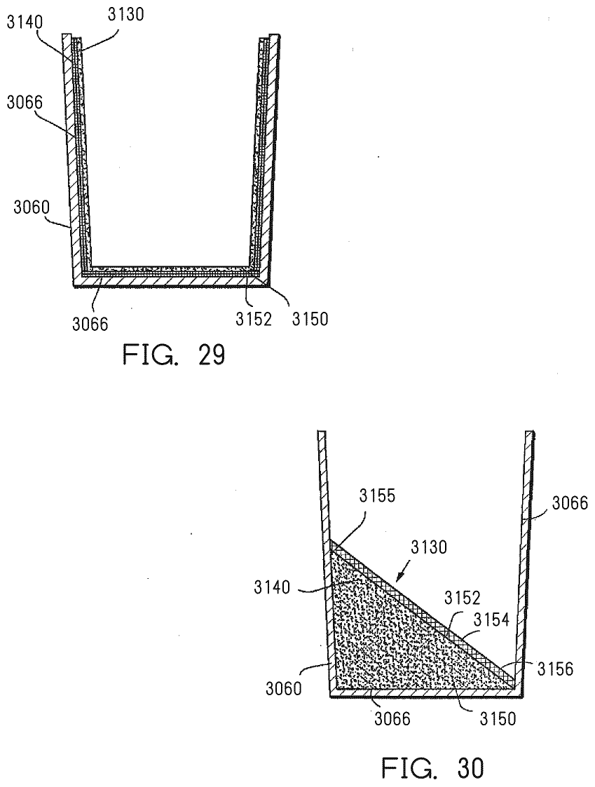

Turning now to FIGS. 29-30, an alternative embodiment of the baffle mechanism 3130 of the present disclosure is illustrated. Baffle mechanism 3130 includes a baffle 3140 in the form of a fluid solidifier 3150 which allows collected wound fluid and exudates to gel or solidify thereby consolidating and containing the exudates in the collection canister 3060. The fluid solidifier 3150 alters the state of the fluids and exudates to a substantially non-flowable state. By rendering the collected exudates in a non-flowable state, the collection canister 3060 may be more portable by dampening exudates movement within the canister 3060. Consolidation may also help avoid vacuum delay by minimizing clogging of filter 3076, preventing inadvertent contact of the exudates with the pressure transducer port 3075, and unintentional triggering of fill sensor 3079.

Fluid solidifier 3150 may be one or a mixture of absorbents having the same or different bulk densities. In embodiments, the fluid solidifier 3150 is in powder form. The absorbents may also have the same or different absorbing capacity. The absorbing capacity may be influenced by the shape of the absorbent and the wettability of the absorbent by the wound exudates, among other things. The rate of absorption may be dependent upon the ratio of the amount of absorbent used per volume of exudates.

The fluid solidifier 3150 may be any commercially available solidifier agent such as Solidifier manufactured by DeRoyal Industries, Absorb-O-Gel™ available from Pioneer Medical, Aqua-Keep™ manufactured by Sumitomo and available from Absorbent Technologies, Norscoryl™ S-35 manufactured by Emerging Technologies, Inc. and available through The Chemical Company, Medigel™ 300 available from BASF, Flosorb™ 60 available from Chemtall, Inc., and SA60N type II, available from Absorbent Technologies. In embodiments the fluid solidifier 3150 may be mixed with a bioactive agent as described above.

Fluid solidifier 3150 may be disposed in any portion of collection canister 3060. As illustrated in the embodiment of FIG. 29, the fluid solidifier 3150 is applied to the walls 3066 of the canister 3060 by use of a water soluble adhesive 3152. The water soluble adhesive 3152 may be any adhesive that allows for temporary bonding of the fluid solidifier 3150 until it is contacted with a fluid. The water soluble adhesive 3152 is of sufficient adhesive strength to maintain the fluid solidifier 3150 on the walls 3066 of the canister 3060 when dry. The adhesive 3152 can be separated from the wall 3066 once wet. Accordingly, after the wound fluid or exudates contacts a portion of wall 3066, the adhesive 3152 releases the fluid solidifier 3150 from the wall 3066.

The water soluble adhesive 3152 may be sprayed or coated onto the walls 3066 of the canister 3060. The fluid solidifier 3150 may then be applied thereon in powder or other form by any suitable technique. This configuration will ration the fluid solidifier 3150 to only be used when wound fluid has activated it, based on the volume of exudates collected therein. Once the exudates contact the wall, the adhesive 3152 releases the fluid solidifier 3150 which in turn absorbs the wound fluid. The fluid solidifier 3150 may be applied uniformly throughout the canister 3060, in select portions therein, or in varying amounts, such as a concentration gradient of more to less from the top to the bottom of the canister 3060.

As an alternative, the fluid solidifier 3150 may be placed in the bottom of canister 3060 as illustrated in FIG. 30 and covered and maintained with a water soluble film 3154. The water soluble film 3154 may be pre-formed or cured from a water soluble adhesive 3152. For example, the water soluble adhesive 3152 may be spread onto a slab and dried, by air or the addition of heat, to form film 3154. The film may be of uniform or varying thickness. In embodiments, an adhesive may be used to fix the ends of the film 3154 to canister 3060. As illustrated in the present embodiment, the fluid solidifier 3150 may be disposed in the canister 3060 at an incline or slope relative to the bottom of canister 3060. Accordingly, the film 3154 may be thicker at a first end 3155 relative to a second end 3156 so that the exudates may flow down the include to the lowest available level of the collection canister 3060 where any fluid solidifier 3150 disposed therein may absorb the wound fluid.

Although embodiments of the present disclosure have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

While the disclosure has been illustrated and described, it is not intended to be limited to the details shown, since various modifications and substitutions can be made without departing in any way from the spirit of the present disclosure. As such, further modifications and equivalents of the invention herein disclosed can occur to persons skilled in the art, and all such modifications and equivalents are believed to be within the spirit and scope of the disclosure as defined by the following claims.

1.-46. (canceled)

47. A negative pressure wound therapy system, comprising:
a negative pressure source configured to aspirate fluid from a wound covered by a wound dressing;
a canister comprising an interior volume configured to store fluid aspirated by the negative pressure source; and
a sensor configured to detect a particular volume of fluid collected in the interior volume of the canister, the sensor comprising two electrodes supported by the canister and configured to contact the fluid in the interior volume of the canister,
wherein a portion of each of the two electrodes extends a predetermined distance from a top surface of the canister into the internal volume of the canister,
wherein the predetermined distance from the top surface of the canister corresponds to the particular volume of fluid collected in the interior volume of the canister, and
wherein a change in an electrical property associated with the two electrodes indicates that the particular volume of fluid has been stored in the interior volume of the canister.

48. The negative pressure wound therapy system of claim 47, further comprising a baffle unit disposed inside the canister.

49. The negative pressure wound therapy system of claim 48, wherein the baffle unit at least partially surrounds the two electrodes.

50. The negative pressure wound therapy system of claim 48, wherein the baffle unit comprises a first baffle member and a second baffle member, the first and second baffle members supported by the canister and extending from the top surface of the canister into the internal volume of the canister.

51. The negative pressure wound therapy system of claim 50, wherein a length of the first baffle member is greater than a length of the second baffle member.

52. The negative pressure wound therapy system of claim 47, wherein at least one of the two electrodes comprises a coating, the coating configured to dissolve when the coating is in contact with the fluid collected in the interior volume of the canister for a predetermined time.

53. The negative pressure wound therapy system of claim 52, wherein the coating is configured to inhibit encrustation formed by the drying of the fluid collected in the interior volume of the canister.

54. The negative pressure wound therapy system of claim 52, wherein the coating comprises a water-soluble coating.

55. The negative pressure wound therapy system of claim 54, wherein the predetermined time depends on at least one of a characteristic of the water soluble coating, a characteristic of the fluid collected in the interior volume of the canister, and a rate of flow of the fluid into the canister.

56. The negative pressure wound therapy system of claim 55, wherein the characteristic of the water soluble coating is at least one of a thickness of the water soluble coating, a density of the water soluble coating, and a composition of the water soluble coating.

57. The negative pressure wound therapy system of claim 55, wherein the characteristic of the fluid collected in the interior volume of the canister is at least one of a pH of the fluid, a viscosity of the fluid, and an ionic composition of the fluid.

58. The negative pressure wound therapy system of claim 47, further comprising a circuit configured to detect the change in the electrical property associated with the two electrodes, wherein the change in the electrical property comprises a change in at least one of resistance, capacitance, or voltage.

59. The negative pressure wound therapy system of claim 58, wherein the change in the electrical property associated with the two electrodes is used to activate an indicator for indicating a condition.

60. The negative pressure wound therapy system of claim 47, wherein the particular volume of fluid that has been collected in the canister comprises a volume associated with a replace-canister condition.

61. The negative pressure wound therapy system of claim 47, wherein the particular volume of fluid that has been collected in the canister comprises a volume associated with a full-canister condition.
